(19) United States
(12) Patent Application Publication
(10) Pub. No.: US 2024/0173293 A1
(43) Pub. Date: May 30, 2024

(54) Treatment of Breast Cancer with Amcenestrant and Palbociclib

(71) Applicants: Sanofi, Paris (FR); Pfizer Inc., New York, NY (US)

(72) Inventors: Marina CELANOVIC, Newton, MA (US); Gautier PAUX, Cambridge, MA (US); Justin Thomas HOFFMAN, San Diego, CA (US); Sandrine ANNEHEIM, Paris (FR); Sylvaine CARTOT-COTTON, Acheres la Foret (FR); Patrick COHEN, Paris (FR); Alice GOSSELIN, Paris (FR)

(73) Assignees: Sanofi, Paris (FR); Pfizer Inc., New York, NY (US)

(21) Appl. No.: 18/549,158

(22) PCT Filed: Mar. 2, 2022
(86) PCT No.: PCT/US2022/018564

§ 371 Date: Sep. 5, 2023

Related U.S. Application Data
(60) Provisional application No. 63/286,457, filed on Dec. 6, 2021.
(60) Provisional application No. 63/242,869, filed on Sep. 10, 2021.
(60) Provisional application No. 63/190,151, filed on May 18, 2021.
(60) Provisional application No. 63/155,687, filed on Mar. 2, 2021.

(30) Foreign Application Priority Data
Dec. 9, 2021 (EP) 21315270.5

(51) Int. Cl. A61K 31/40; A61K 9/00; A61K 31/519; A61P 35/04

(52) U.S. Cl. CPC A61K 31/40; A61K 9/0053; A61K 31/519; A61P 35/04

(57) ABSTRACT

The present disclosure provides methods of treating breast cancer with a combination of amcenestrant and palbociclib.

### CROSS-REFERENCE TO RELATED APPLICATIONS

The present application claims priority from U.S. Applications 63/155,687, filed Mar. 2, 2021; 63/190,151, filed May 18, 2021; 63/242,869, filed Sep. 10, 2021; and 63/286,457, filed Dec. 6, 2021. The present application also claims priority to EP Application 21315270.5, filed Dec. 9, 2021. The contents of the priority applications are incorporated by reference herein in their entirety.

### BACKGROUND OF THE INVENTION

Breast cancer is the most commonly diagnosed cancer and the second leading cause of death in women in the United States. Both endogenous and exogenous steroid hormones such as estrogen and progesterone have been implicated in the pathogenesis of breast cancer. Clinical treatment decisions are driven by the expression status of estrogen receptors (ERs), progesterone receptors (PgRs), and human epidermal growth factor receptor 2 (HER2). There are three clinical subtypes of breast cancer: HER2+, ER+/HER2− and ER−/PgR−/HER2− (triple negative).

A key protein target in treatment of breast cancer is estrogen receptor alpha (ERα), a hormone regulator transcription factor that is encoded by the ESR1 gene and expressed at elevated levels in approximately about 75% of breast tumors. ERα enables breast tumors to respond to the mitogenic actions of estrogens. ER-positive breast cancers respond well to therapy targeting ER signaling either through competitive binding of ER by antagonists or by blocking the production of estrogen by aromatase inhibitors (AIs). Sequential hormonal therapy (alone or in combination) is currently the standard of care in the metastatic breast cancer setting for ER+/HER2− patients without rapidly progressing visceral or symptomatic metastases. Common classes of drugs used for this purpose include selective estrogen receptor modulators (SERMs) such as tamoxifen; AIs such as letrozole, anastrozole, and exemestane; selective estrogen receptor down-regulators or degraders (SERDs) such as fulvestrant; and luteinizing hormone releasing hormone agonists such as buserelin and goserelin.

Unfortunately, not all patients respond to first-line hormonal therapy as they present with primary or de novo resistance, and some patients who initially respond subsequently have breast cancer progression (acquired resistance). Resistance to endocrine therapies is frequent but relapsed tumors remain dependent on ER, which is highlighted by patient responses to second- and third-line endocrine therapies after failure of an earlier line of hormonal therapy. Estrogen receptor alpha signaling reactivation can occur due to change in ligand sensitivity and specificity or by new mutations of the estrogen receptor 1 (ESR1). Estrogen receptor 1 gene mutation was recently evaluated in clinical studies with a high prevalence (25% to 40%) in relapse patients after AI therapy; with a limited benefit with current monotherapy.

SERDs are competitive ER antagonists that also induce conformational changes that lead to the degradation of ER via a ubiquitin-proteasome system. The unique dual-function of SERDs (ER antagonism and depletion) may enable them to block ER signaling in cellular settings where other endocrine agents, such as tamoxifen or AIs have failed. Although fulvestrant may have served as a proof of concept for the SERD approach, this therapy is limited by its poor pharmaceutical properties, which necessitate intramuscular administration and limit the applied dose, exposure and receptor engagement. Patients treated with the fulvestrant 500 mg regimen (500 mg on Days 1, 14, 28; monthly thereafter) exhibited improvement in progression free survival and overall survival over the initially marketed 250 mg dose. However, the 500 mg dose of fulvestrant does not fully saturate ER binding in patients as inhibition of ¹⁸F-fluorestradiol (18FES) positron emission tomography (PET) scan uptake was incomplete in 38% of the treated patients. This lack of receptor occupancy was associated with lack of clinical benefit.

Thus, there remains a need for an effective and well-tolerated SERD therapy with improved route of administration (oral versus intramuscular route), bioavailability, and maintenance of ER receptor blockade.

### SUMMARY OF THE INVENTION

The present disclosure provides a method of treating breast cancer in a patient in need thereof, comprising orally administering to the patient amcenestrant, or a pharmaceutically acceptable salt thereof, at a dose of 200-400 mg once daily and orally administering to the patient palbociclib, or a pharmaceutically acceptable salt thereof, at a dose of 75-125 mg once daily.

In some embodiments, the daily dose of amcenestrant is 200 mg. In some embodiments, the daily dose of palbociclib is 75, 100, or 125 mg. In certain embodiments, the daily dose of palbociclib is 125 mg.

In some embodiments, amcenestrant and palbociclib or a pharmaceutically acceptable salt thereof are administered to the patient in one or more treatment cycles (e.g., 1, 2, 3, 4, 5, 6, 7, 8, 9, 10, 11, 12, 13, 14, or more treatment cycles). In further embodiments, each of the one or more treatment cycles comprises or consists of 28 days. In certain embodiments, the patient is administered amcenestrant daily, and is administered palbociclib, or a pharmaceutically acceptable salt thereof, daily for 21 consecutive days only, during each of the one or more treatment cycles. In certain embodiments, a treatment cycle is followed immediately by a subsequent treatment cycle if more than one treatment cycle is administered. In particular embodiments, the patient is treated with amcenestrant daily at 200 mg per day, and with palbociclib at 125 mg daily for 21 consecutive days only, in one or more 28-day treatment cycles.

In some embodiments, the breast cancer the patient has is hormone receptor (HR) positive, for example, ER-positive and/or PgR-positive. In further embodiments, the breast cancer is HER2-negative. In certain embodiments, the breast cancer is ER+/HER2−. In some embodiments, the breast cancer is advanced or metastatic cancer. In further embodiments, the patient is a postmenopausal woman with ER+/HER2− advanced or metastatic breast cancer.

In some embodiments, an ESR1 gene in the patient is mutated. In further embodiments, the ESR1 gene in the patient contains an E380Q, Y537N/S, and/or D538G mutations.

In some embodiments, the patient is refractory to endocrine therapy.

In some embodiments, the patient has not previously been treated with a CDK4/6 or mTOR inhibitor.

In some embodiments, the present treatment results in partial response (PR) or stable disease (SD) in the patient. In some embodiments, the present treatment results in progression-free survival (PFS), more particularly a median PFS of about 14.7 months, in the patient.

In some embodiments, the present treatment method further comprises monitoring the patient for neutropenia or lung inflammation.

In some embodiments, amcenestrant or a pharmaceutically acceptable salt thereof is provided as a capsule or a tablet to the patient.

In some embodiments, palbociclib or a pharmaceutically acceptable salt thereof is provided as a capsule or a tablet to the patient.

In another aspect, the present disclosure provides an article of manufacture or kit, comprising an amcenestrant capsule or tablet at 200 mg per capsule or tablet and a palbociclib capsule or tablet at 125 mg per capsule or tablet. In some embodiments, the article of manufacture or kit comprises 28 of the amcenestrant capsules or tablets and 21 of the palbociclib capsules or tablets, and optionally instructions for use for treating ER+/HER2− breast cancer.

Provided herein are palbociclib or a pharmaceutically acceptable salt thereof for use in combination with amcenestrant to treat breast cancer in a method herein; and amcenestrant for use in combination with palbociclib or a pharmaceutically acceptable salt thereof to treat breast cancer in a method herein. Also provided are use of palbociclib or a pharmaceutically acceptable salt thereof in the manufacture of a medicament to be used in combination with amcenestrant to treat breast cancer in the present method; and use of amcenestrant in the manufacture of a medicament to be used in combination with palbociclib or a pharmaceutically acceptable salt thereof to treat breast cancer in the present method.

Other features, objectives, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a diagram showing the clinical studies undertaken to determine the optimal dose of amcenestrant in combination with palbociclib.

FIG. 2 is a bar graph showing the BOR (Best Overall Response) and best relative change from baseline in tumor size in patients in the “Pooled Population Activity” population from a patient database extraction with a cut-off date of Mar. 30, 2021. Each bar represents a single patient, with stars highlighting the ongoing patients still undergoing treatment at the time of the database extraction. Best overall response is described as follows: partial response (PR), stable disease (SD), progressive disease (PD). Best relative change is presented as percent change in tumor size compared to baseline, with circles representing patients with 0% tumor shrinkage. BOR and best relative change were assessed by investigators/local radiologists.

FIG. 3 is a bar graph showing the BOR and best relative change from baseline in tumor size in patients in the “Pooled Population Activity” population from a patient database extraction with a cut-off date of Oct. 4, 2021. Labeling conventions are as described for FIG. 2.

FIG. 4 is a chart showing the antitumor activity of combination treatment in the response-evaluable population (34 patients) and subgroups depending on prior therapy and baseline ESR1 mutation status.

### DETAILED DESCRIPTION OF THE INVENTION

The present disclosure provides a combination therapy with amcenestrant and palbociclib for treatment of breast cancer. Palbociclib is a kinase inhibitor indicated for the treatment of adult patients with hormone receptor (HR)-positive, HER2-negative advanced or metastatic breast cancer in combination with an aromatase inhibitor as initial endocrine-based therapy in postmenopausal women or in men; or with fulvestrant in patients with disease progression following endocrine therapy. The present inventors have discovered that amcenestrant, a SERD, exhibits a favorable overall safety profile when used at a 200 mg dose in combination with palbociclib in patients with ER+/HER2− advanced breast cancer. The combination regimen described herein has been shown to achieve an Objective Response Rate (ORR) of 32.4% in hormonal resistant patients, with partial responses (PR) observed in 32.4% of patients and stable disease (SD) observed in 64.7% of patients. The clinical benefit rate (CBR) has been measured at 73.5% and the disease control rate (DCR) at 97.1%. Tumor shrinkage was observed in 79.4% of the patients, amongst which two patients showed 100% shrinkage of target lesions. The clinical benefit has been observed in almost all patients with ESR1 mutation (7/8 patients, CBR of 87.5%) and in 18 out of the 26 patients with wild-type ESR1 (CBR of 69.2%).

The favorable safety profile of the combination regimen described herein is further detailed below. Furthermore, compared to previous combination therapy with fulvestrant and palbociclib, the present regimen is completely oral-based. Thus, the dosing regimen herein provides a safe, active, and convenient treatment for ER+ breast cancer patients, including those who have failed other therapy, such as endocrine therapy.

### I. THERAPEUTIC AGENTS

A. Amcenestrant

Amcenestrant is a potent, orally bioavailable, and selective ER inhibitor that belongs to the SERD family of compounds. Amcenestrant has complete estrogen receptor antagonist properties and accelerates the proteasomal degradation of ER. Amcenestrant (aka. SAR439859) has the chemical name 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid, or 8-(2,4-dichlorophenyl)-9-(4-{[(3S)-1-(3-fluoropropyl)pyrrolidin-3-yl]oxy}phenyl)-6,7-dihydro-5Hbenzo[7]annulene-3-carboxylic acid (C₃₁H₃₀C₁₂FNO₃). See also WO 2017/140669. Amcenestrant has the following structural formula (I):

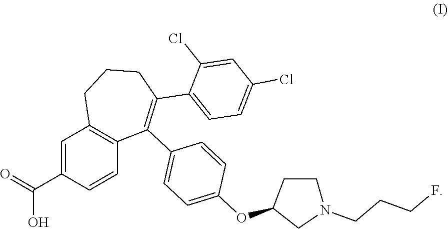 [SMILES: O=C(O)c1ccc2c(c1)CCCC(c1ccc(Cl)cc1Cl)=C2c1ccc(O[C@H]2CCN(CCCF)C2)cc1]

Amcenestrant may be provided as a zwitterion (i.e., a globally neutral molecule having one acidic and one basic group), with no additional counterions. Amcenestrant may also be provided in the form of a salt with one or more additional acidic or basic molecule(s). Unless otherwise noted, the dose of amcenestrant administered to a patient refers to the dose of free zwitterionic (i.e., uncharged) amcenestrant administered, and does not include the weight of any counterions.

As used herein, amcenestrant may be provided in a pharmaceutical composition comprising amcenestrant or a pharmaceutically acceptable salt thereof, with or without other active ingredients. The pharmaceutical composition may typically be in the form of, e.g., a liquid solution, dispersion, suspension, tablet, capsule, or the like. The pharmaceutical composition may comprise inactive ingredients that are pharmaceutically acceptable excipients and/or carriers. Amcenestrant is typically administered orally. Patients may take the medication with food or without food.

In some embodiments, amcenestrant is suitable for oral administration formulated as a capsule comprising 100 mg of amcenestrant.

B. Palbociclib

Palbociclib is an inhibitor of the cyclin-dependent kinases CDK4 and CDK6. Palbociclib is marketed under the tradename Ibrance®, and may also be referred to as 6-acetyl-8-cyclopentyl-5-methyl-2-{[5-(piperazin-1-yl)pyridin-2-yl]amino}pyrido[2,3-d]pyrimidin-7(8H)-one. The molecular formula of palbociclib is C₂₄H₂₉N₇O₂. Its structural formula is shown below:

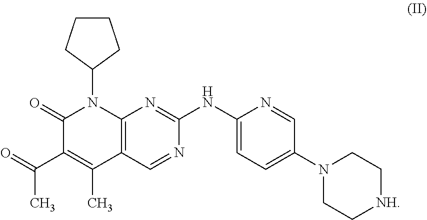 [SMILES: CC(=O)c1c(C)c2cnc(Nc3ccc(N4CCNCC4)cn3)nc2n(C2CCCC2)c1=O]

See also WO 2003/062236.

Palbociclib may be a yellow to orange powder with pKa of 7.4 (the secondary piperazine nitrogen) and 3.9 (the pyridine nitrogen). At or below pH 4, palbociclib behaves as a high-solubility compound. Above pH 4, the solubility of the drug substance reduces significantly.

As used herein, palbociclib is provided in a pharmaceutical composition comprising palbociclib or a pharmaceutically acceptable salt thereof, with or without other active ingredients. The pharmaceutical composition may be in the form of, e.g., a liquid solution, dispersion, suspension, tablet, capsule, or the like. The pharmaceutical composition may comprise inactive ingredients that are pharmaceutically acceptable excipients and/or carriers. Palbociclib is typically administered orally. Patients may take the medication with food or without food, according to the label for the approved capsule or tablet formulation, respectively. In some embodiments, patients may take the medication with food when palbociclib is provided in the form of a capsule.

In some embodiments, palbociclib is suitable for oral administration formulated as a capsule or tablet at a strength of 125 mg, 100 mg, or 75 mg of palbociclib per capsule or tablet. In some embodiments, a palbociclib capsule may comprise one or more, e.g., all, of the following inactive ingredients: microcrystalline cellulose, lactose monohydrate, sodium starch glycolate, colloidal silicon dioxide, magnesium stearate, and hard gelatin capsule shells. In some embodiments, a palbociclib tablet may comprise one or more, e.g., all of the following inactive ingredients: microcrystalline cellulose, colloidal silicon dioxide, crospovidone, magnesium stearate, succinic acid, HPMC 2910/hypromellose, titanium dioxide, triacetin, and coloring agents (e.g., FD&C Blue #2, Indigo Carmine Aluminum Lake, red iron oxide, or yellow iron oxide).

Palbociclib may be provided as a free base form with no additional counterions, or may be provided in the form of a salt with one or more additional molecule(s). Unless otherwise noted, the dose of palbociclib administered to a patient refers to the dose of free base (i.e., uncharged) palbociclib administered, and does not include the weight of any counterions.

### II. TREATMENT REGIMENS

The patient may receive a treatment regimen of the present disclosure for at least 4, 8, 12, 16, 20, or 24 weeks or longer; until the patient no longer benefits from the treatment, shows disease progression, or shows unacceptable toxicity.

As used herein, administration of amcenestrant or palbociclib includes self-administration by the patient (e.g., oral intake by the patient).

In some embodiments, amcenestrant is administered in a total daily amount of 200 or 400 mg. Amcenestrant may be administered orally once a day (QD) (i.e., once every 24 hours). In some embodiments, amcenestrant is administered in the morning, at approximately the same time each day (±three hours). A capsule or tablet comprising amcenestrant may be swallowed whole with a glass of non-carbonated water. After ingesting amcenestrant, the patient may ingest a second glass of water which may also to be used to rinse out the mouth. In some embodiments, the capsule is swallowed with a total of approximately 240 mL water.

In some embodiments, palbociclib or a pharmaceutically acceptable salt thereof is administered to patients in one or more treatment cycles, each cycle having 21 consecutive days (i.e., three weeks) on treatment, followed by seven days (i.e., one week) off treatment (no palbociclib); thus, each treatment cycle has 28 days (i.e., four weeks). During on-treatment days in the cycle, the patient may take a single dose of palbociclib or a pharmaceutically acceptable salt thereof once daily (QD) orally. In some embodiments, palbociclib or a pharmaceutically acceptable salt thereof is taken at approximately the same time each day (±three hours). Palbociclib or a pharmaceutically acceptable salt thereof may be ingested as a capsule or tablet comprising 75 mg, 100 mg, or 125 mg of palbociclib. For clarity, the 75 and 100 mg doses may be administered to a patient should the patient experience toxicities and/or adverse events when treated with the 125 mg dose, or, as contemplated in a palbociclib label, as per the patient profile and concomitant therapies. In some embodiments, palbociclib is not ingested with grapefruit juice.

In some embodiments, the patient may be treated with an amcenestrant/palbociclib combination therapy in one or more treatment cycles, each treatment cycle being 4 weeks (28 days) long. During each treatment cycle (“4w” cycle), the patient may take amcenestrant at 200 or 400 mg QD, while taking palbociclib or a pharmaceutically acceptable salt thereof at 75, 100, or 125 mg QD for three consecutive weeks (21 consecutive days) followed by one week (7 days) off treatment (no palbocilicb) (“3w/1w”). In certain embodiments, the patient is treated with amcenestrant at 200 mg QD and palbociclib at 125 mg QD 3w/1w in one or more 4w treatment cycles. In certain embodiments, the patient is treated with amcenestrant at 400 mg QD and palbociclib at 125 mg QD 3w/1w in one or more 4w treatment cycles. On days when both medications are taken, the two medications may be taken together or sequentially. For example, the two medications may be taken more than 30 minutes apart, e.g., more one hour, three hours, six hours, or 12 hours apart.

In certain embodiments, the patient may be treated with one, two, three, four, five, six, seven, eight, nine, ten, eleven, twelve, or more treatment cycles (i.e., 4w cycles of amcenestrant QD/palbociclib QD 3w/1w). In some embodiments, the treatment is continued until the patient no longer benefits from the treatment. In some other embodiments, the treatment continues until the patient shows unacceptable toxicity. In some other embodiments, the treatment continues until the patient shows disease progression.

During the treatment period, the patient is monitored regularly for disease status and/or for dose adjustment.

During the treatment period (i.e., the one or more treatment cycles), the patient may advantageously also be monitored for possible side effects, including toxicities and adverse events. In particular, a dose modification (e.g., dose interruption or reduction, or delay of the start of a treatment cycle) in palbociclib or a pharmaceutically acceptable salt thereof may be implemented in case of hematologic toxicities (e.g., neutropenia), concomitant use of a strong CYP3A inhibitor, or hepatic impairment, as per palbociclib label. For example, the blood cell count and lung functions of the patient may be monitored for possible occurrence of neutropenia and lung problems such as lung inflammation (e.g., interstitial lung disease or pneumonitis). Complete blood count of the patient may be measured prior to start of the combination therapy, at the beginning of each cycle, as well as during the cycle (e.g., on Day 15 of the first two cycles), and as clinically indicated.

During the treatment period, the patient may be advised to avoid concurrent use of strong CYP3A inhibitors or inducers.

### III. SELECTION OF PATIENTS

The present combination therapy may be used on hormone receptor (HR)-positive breast cancer patients. In some embodiments, the patient has ER+/HER2− advanced or metastatic breast cancer. In further embodiments, the patient is a postmenopausal woman with ER+/HER2− advanced or metastatic breast cancer. In certain embodiments, the present combination therapy may be used to treat ER+/HER2− advanced or metastatic breast cancer in postmenopausal women, or in patients (e.g., adult patients, ≥18 years of age) with disease progression following endocrine therapy. In further embodiments, the therapy for such patients comprises oral administration of amcenestrant at 200 mg and palbociclib at 125 mg QD in one or more 3w/1w treatment cycles.

A postmenopausal woman may be a woman who is greater than 60 years of age; or a woman who is ≤60 years of age, who also has:
(i) had spontaneous cessation of menses >12 months prior to registration, in the absence of chemotherapy, tamoxifen, and/or toremifene treatment;
(ii) had cessation of menses of duration ≤12 months prior to registration or secondary to hysterectomy, and who also has a follicle stimulating hormone (FSH) level in the postmenopausal range, according to institutional standards;
(iii) received hormonal replacement therapy but who has discontinued this treatment, and who also has an FSH level in the postmenopausal range according to institutional standards;
(iv) undergone a bilateral surgical oophorectomy; and/or
(v) been treated with a gonadotropin-releasing hormone (GnRH) analog for at least six months, and has had a negative pregnancy test prior to initiation of study treatment and at monthly intervals during treatment.
   In some embodiments, the postmenopausal range for FSH levels is >34.4 IU/L.

In some embodiments, the patient has a histologically or cytologically proven diagnosis of the breast adenocarcinoma with evidence of inoperability, metastatic disease, and/or locally advanced cancer not amenable to radiation therapy or surgery in a curative intent.

In some embodiments, the patient has a primary tumor or any metastatic site that is positive for ER and does not also overexpress HER2. A primary tumor or any metastatic site is judged to be positive for ER when >1% of the tumor cells are stained by immunohistochemistry (IHC). A primary tumor or any metastatic site can be judged to not be overexpressing HER2 by using, for example, IHC (0, 1+) or in situ hybridization (negative based on single-probe average HER2 copy number <4.0 signals/cell, or dual-probe HER2/centromeric probe for chromosome 17 (CEP17) ratio <2 with an average HER2 copy number <4.0 signals/cell, as per the American Society of Clinical Oncology guidelines).

In some embodiments, the patient has a measurable lesion or measurable malignant lymph node by the RECIST v1.1 criteria (Response Evaluation Criteria In Solid Tumors, see Eisenhauer et al. Eur J Cancer. (2009) 45(2):228-47). A measurable lesion refers to a tumor lesion that has been accurately measured in at least one dimension (in the plane of measurement is to be recorded) with a minimum longest diameter of:
(i) 10 mm by CT scan (CT scan slice thickness no greater than 5 mm);
(ii) 10 mm caliper measurement by clinical exam (lesions which cannot be accurately measured with calipers should be recorded as nonmeasurable); or
(iii) 20 mm by chest X-ray.
   A measurable malignant lymph node is a lymph node that is pathologically enlarged and measurable. A malignant lymph node must be ≥15 mm in short axis when assessed by CT scan (CT scan slice thickness recommended to be no greater than 5 mm).

In some embodiments, the patient has undergone a prior chemotherapeutic regimen for advanced/metastatic disease. Prior chemotherapy treatment includes treatment with antibody drug conjugates (ADCs). In some embodiments, the patient has received no more than one prior chemotherapeutic regimen. In some embodiments, the patient has received at least six months of prior endocrine therapy for advanced breast cancer. In some embodiments, the patient has undergone no more than two prior lines of endocrine therapy. In some embodiments, the patient is a patient who has previously had early progression on adjuvant endocrine therapy, or a patient who progressed on adjuvant endocrine therapy within twelve months after completion of the treatment period.

### IV. TREATMENT OUTCOMES AND TUMOR RESPONSE EVALUATION

The present combination therapy may result in a complete response (CR), partial response (PR), or stable disease (SD) in patients, and may prevent progressive disease (PD). Bioimaging can be used to evaluate the level of response to the therapy. Cytology and histology may also be used as needed (e.g., to locate any residual lesions). In some embodiments, the various levels of response can be evaluated in accordance with Tables 1 and 2 below.

TABLE 1
Evaluation of Target Lesions

| Response Classification | Criteria |
|---|---|
| Complete Response (CR) | Disappearance of all target lesions. Any pathological lymph nodes |
| | (whether target or nontarget) must have reduction in short axis to <10 |
| | mm. |
| Partial Response (PR) | At least a 30% decrease in the sum of diameters of target lesions, |
| | taking as reference the baseline sum diameters. |
| Progressive Disease (PD) | At least a 20% increase in the sum of diameters of target lesions, taking |
| | as reference the smallest sum on study (this includes the baseline sum |
| | if that is the smallest on study). In addition to the relative increase of |
| | 20%, the sum must also demonstrate an absolute increase of at least 5 |
| | mm. (Note: the appearance of one or more new lesions is also |
| | considered progression). |
| Stable Disease (SD) | Neither sufficient shrinkage to qualify for PR nor sufficient increase to |
| | qualify for PD, taking as reference the smallest sum diameters while on |
| | study |

TABLE 2
Evaluation of Non-Target Lesions

| Response Classification | Criteria |
|---|---|
| Complete Response (CR) | Disappearance of all nontarget lesions and normalization of tumor |
| | marker level. All lymph nodes must be nonpathological in size (<10 |
| | mm short axis). |
| Incomplete Response/ | Persistence of one or more nontarget lesion(s) and/or maintenance of |
| Stable Disease (SD) | tumor marker level above the normal limits. |
| Progressive Disease (PD) | Unequivocal progression (see comments below) of existing nontarget |
| | lesions. (Note: the appearance of one or more new lesions is also |
| | considered progression). |

The time to first tumor response (CR or PR) is defined as the time interval from the date of first administration of the study treatment to the date of the first occurrence of CR or PR (that is subsequently confirmed). The objective response rate (ORR) is determined as per RECIST v1.1; it corresponds to confirmed CR or PR. The clinical benefit (CR+PR+SD≥24 weeks), expressed as the clinical benefit rate (CBR), can be assessed as per RECIST v1.1 (Eisenhauer et al., Eur J Cancer. (2009) 45(2):228-47). The disease control rate (DCR) corresponds to CR, PR, or SD. A PR or a CR is confirmed on a second examination done at least four weeks apart in order to confirm the antitumoral response.

In order to evaluate best overall response (BOR), if a CR is truly met at the first time point, then any disease seen at a subsequent time point, even disease meeting PR criteria relative to baseline, makes the disease PD at the second time point (since the disease reappeared after the CR). Best response depends on whether the minimum duration for SD was met. However, sometimes “CR” may be claimed when subsequent scans suggest that small lesions are likely still present and in fact the patient had PR, not CR, at the first time point. Under these circumstances, the original CR is changed to PR and the best response is PR. The best overall response may be determined once all the data for the patient is known. In some embodiments, criteria to determine the overall response and best overall response may be evaluated in accordance with Tables 3 and 4, respectively.

TABLE 3
Evaluation of Overall Response

| Target lesions | Non-target lesions | New lesions | Overall response |
|---|---|---|---|
| CR | CR | No | CR |
| CR | Non-CR/non-PD | No | PR |
| CR | Not evaluated | No | PR |
| PR | Non-PD or | No | PR |
| | not all evaluated | | |
| SD | Non-PD or | No | SD |
| | not all evaluated | | |
| Not all evaluated | Non-PD | No | Not evaluated |
| PD | Any | Yes or No | PD |
| Any | PD | Yes or No | PD |
| Any | Any | Yes | PD |

TABLE 4
Evaluation of Best Overall Response

| Overall | Overall | |
|---|---|---|
| response | response | |
| First | Subsequent | |
| time point | time point | Best overall response |
| CR | CR | CR |
| CR | PR | SD, PD or PR |
| CR | SD | SD provided minimum criteria for SD |
| | | duration met; otherwise, PD |
| CR | PD | SD provided minimum criteria for SD |
| | | duration met; otherwise, PD |
| CR | NE | SD provided minimum criteria for SD |
| | | duration met; otherwise not evaluated |
| | | (NE) |
| PR | CR | PR |
| PR | PR | PR |
| PR | SD | SD |
| PR | PD | SD provided minimum criteria for SD |
| | | duration met, otherwise, PD |
| PR | NE | SD provided minimum criteria for SD |
| | | duration met, otherwise NE |
| NE | NE | NE |

In some embodiments, ORR and clinical benefit rate (CBR) are assessed in patients based on their ESR1 status (mutated or wild type). ESR1 status may be determined by, e.g., using multiplex droplet digital polymerase chain reaction (ddPCR) after extraction of plasma circulating DNA.

In some embodiments, the present therapy (e.g., 200 mg QD amcenestrant/125 mg QD 3w/1w palbociclib in one or more 4w treatment cycles) leads to an objective response in at least 20%, e.g., at least 21, 22, 23, 24, 25, 26, 27, 28, 29, or 30% of treated patients. In further embodiments, the therapy achieves this result in postmenopausal women with ER+/HER2− advanced or metastatic breast cancer.

In some embodiments, the present therapy (e.g., 200 mg QD amcenestrant/125 mg QD 3w/1w palbociclib in one or more 4w treatment cycles) leads to PR in at least 20%, e.g., at least 21, 22, 23, 24, 25, 26, 27, 28, 29, or 30%, of treated patients. In further embodiments, the therapy achieves this result in postmenopausal women with ER+/HER2− advanced or metastatic breast cancer.

In some embodiments, the present therapy (e.g., 200 mg QD amcenestrant/125 mg QD 3w/1w palbociclib in one or more 4w treatment cycles) leads to SD in at least 50%, e.g., at least 55, 60, 61, or 62%, of treated patients. In further embodiments, the therapy achieves this result in postmenopausal women with ER+/HER2− advanced or metastatic breast cancer.

In some embodiments, the present therapy (e.g., 200 mg QD amcenestrant/125 mg QD 3w/1w palbociclib in one or more 4w treatment cycles) provides clinical benefit in at least 60% (CBR), e.g., at least 65, 70, 71, 72, 73, or 74%, of treated patients. In another embodiment, said present therapy provides clinical benefit in patients irrespective of their ESR1 mutation status. In further embodiments, the CBR in patients with ESR1 mutations is at least 80%, e.g., at least 85, 86, or 87%. In further embodiments, the CBR in patients with wildtype ESR1 is at least 60%, e.g., at least 65, 66, 67, 68, 69, or 70%. In further embodiments, the therapy achieves one of these CBRs in postmenopausal women with ER+/HER2− advanced or metastatic breast cancer.

In some embodiments, the present therapy (e.g., 200 mg QD amcenestrant/125 mg QD 3w/1w palbociclib in one or more 4w treatment cycles) provides disease control in at least 80%, e.g., at least 85, 90, 95, 96, or 97% of treated patients. In further embodiments, the therapy achieves this result in postmenopausal women with ER+/HER2− advanced or metastatic breast cancer.

In some embodiments, the present therapy (e.g., 200 mg QD amcenestrant/125 mg QD 3w/1w palbociclib in one or more 4w treatment cycles) results in a median progression-free survival of, e.g., about 12 to 15, or more months, such as about 14.7 months.

### V. ARTICLES OF MANUFACTURE AND KITS

The present disclosure also provides articles of manufacture, e.g., kits, comprising one or more dosages of amcenestrant and/or palbociclib or a pharmaceutically acceptable salt thereof, and instructions for patients (e.g., for treatment in accordance with a method described herein). Amcenestrant and/or palbociclib tablets or capsules may be blistered and then carded. In some embodiments, each amcenestrant capsule or tablet contains 100 mg of amcenestrant. In some embodiments, each palbociclib capsule or tablet contains 75, 100, or 125 mg of palbociclib. The present disclosure also includes methods for manufacturing said articles. Amcenestrant and palbociclib or a pharmaceutically acceptable salt thereof can be packaged together or separately in suitable packing.

In some embodiments, each package or kit contains 28 capsules or tablets of amcenestrant at a dose strength of 200 mg per capsule or tablet, and 21 capsules or tablets of palbociclib at a dose strength of 125 mg per capsule or tablet. The present invention also includes methods for manufacturing said articles.

### VI. EXEMPLARY EMBODIMENTS

The present disclosure provides a number of exemplary embodiments. Herein is provided a combination comprising 200 mg of amcenestrant, or a pharmaceutically acceptable salt thereof, and palbociclib, or a pharmaceutically acceptable salt thereof, as well as a pharmaceutical composition and kit as described above, for use in the treatment of cancer.

Herein is provided 200 mg of amcenestrant or a pharmaceutically acceptable salt thereof for use in the treatment of cancer by co-administration with palbociclib or a pharmaceutically acceptable salt thereof.

Herein is provided palbociclib or a pharmaceutically acceptable salt thereof for use in the treatment of cancer by co-administration with 200 mg of amcenestrant or a pharmaceutically acceptable salt thereof.

In another embodiment, the cancer is a hormone dependent cancer.

In another embodiment, the cancer is an estrogen receptor dependent cancer, particularly the cancer is an ERα-dependent cancer.

Herein is provided a method of treating the pathological conditions indicated above, particularly breast cancer, comprising administering to a subject in need thereof a pharmaceutical composition as described above.

Herein is provided a method of treating the pathological conditions indicated above, particularly breast cancer, comprising administering to a subject in need thereof a combination as described above.

Herein is provided a method of treating the pathological conditions indicated above, particularly breast cancer, comprising co-administering to a subject in need thereof 200 mg of amcenestrant or a pharmaceutically acceptable salt thereof and palbociclib or a pharmaceutically acceptable salt thereof.

Herein is provided a method of treating the pathological conditions indicated above, particularly breast cancer, comprising co-administering to a subject in need thereof palbociclib, or a pharmaceutically acceptable salt thereof, and 200 mg of amcenestrant or a pharmaceutically acceptable salt thereof.

Herein is provided a method of treating metastatic or advanced breast cancer comprising administering to a patient in need thereof, amcenestrant at a dose of 200 mg per day in combination with palbociclib or a pharmaceutically acceptable salt thereof.

Herein is also provided a method of treating metastatic or advanced breast cancer comprising administering to a patient in need thereof amcenestrant at a dose of 200 mg per day, wherein said patient is also on palbociclib therapy.

Herein is provided a method of treating metastatic or advanced breast cancer comprising administering to a patient in need thereof, amcenestrant at a dose of 200 mg per day in combination with palbociclib or a pharmaceutically acceptable salt thereof.

Herein is provided a method of treating metastatic or advanced breast cancer comprising administering to a patient in need thereof, amcenestrant at a dose of 200 mg per day in combination with palbociclib at a dose of 125 mg per day.

Herein is provided a method of treating metastatic or advanced breast cancer, wherein said method is for a human patient.

Herein is provided a method of treating metastatic or advanced breast cancer, wherein the breast cancer is an ER-positive cancer.

Herein is provided a method of treating metastatic or advanced breast cancer, wherein the breast cancer is a human HER2-negative cancer.

Herein is provided a method of treating metastatic or advanced breast cancer, wherein the breast cancer is an ER-positive, human HER2-negative cancer.

Herein is provided a method of treating metastatic or advanced breast cancer, wherein the patient is a woman.

Herein is provided a method of treating metastatic or advanced breast cancer, wherein the patient is a postmenopausal woman.

Herein is provided a combination comprising 200 mg of amcenestrant, or a pharmaceutically acceptable salt thereof, and palbociclib, or a pharmaceutically acceptable salt thereof, for the manufacture of a medicament useful in treating the pathological conditions indicated above, particularly breast cancer.

Herein is provided the use of amcenestrant, or a pharmaceutically acceptable salt thereof, in the manufacture of a medicament useful in treating the pathological conditions indicated above, particularly breast cancer, by co-administration with palbociclib or a pharmaceutically acceptable salt thereof.

Herein is provided the use of palbociclib, or a pharmaceutically acceptable salt thereof, in the manufacture of a medicament useful in treating the pathological conditions indicated above, particularly breast cancer, by co-administration with amcenestrant or a pharmaceutically acceptable salt thereof.

Herein is provided the use of amcenestrant, or a pharmaceutically acceptable salt thereof, for use in the treatment of metastatic or advanced breast cancer at a dose of 150 to 600 mg per day, in combination with palbociclib or a pharmaceutically acceptable salt thereof.

Herein is provided the use of amcenestrant, or a pharmaceutically acceptable salt thereof, at a dose of 200 mg per day, wherein palbociclib, or a pharmaceutically acceptable salt thereof, is at a dose of 125 mg per day.

Herein is provided the use of amcenestrant, or a pharmaceutically acceptable salt thereof, wherein said use is for a human patient.

Herein is provided the use of amcenestrant, or a pharmaceutically acceptable salt thereof, wherein the breast cancer is an ER-positive cancer.

Herein is provided the use of amcenestrant, or a pharmaceutically acceptable salt thereof, wherein the breast cancer is a human HER2-negative cancer.

Herein is provided the use of amcenestrant, or a pharmaceutically acceptable salt thereof, wherein the breast cancer is an ER-positive, human HER2-negative cancer.

Herein is provided the use of amcenestrant, or a pharmaceutically acceptable salt thereof, wherein the patient is a woman.

Herein is provided the use of amcenestrant, or a pharmaceutically acceptable salt thereof, wherein the patient is a postmenopausal woman.

Herein is provided a combination comprising amcenestrant, or a pharmaceutically acceptable salt thereof, and palbociclib, or a pharmaceutically acceptable salt thereof, for the manufacture of a medicament useful in treating the pathological conditions indicated above, particularly breast cancer.

Herein is provided the use of amcenestrant, or a pharmaceutically acceptable salt thereof, in the manufacture of a medicament useful in treating the pathological conditions indicated above, particularly breast cancer, by co-administration with palbociclib or a pharmaceutically acceptable salt thereof.

Herein is provided the use of palbociclib, or a pharmaceutically acceptable salt thereof, in the manufacture of a medicament useful in treating the pathological conditions indicated above, particularly breast cancer, by co-administration with amcenestrant or a pharmaceutically acceptable salt thereof.

Herein is provided an article of manufacture, a packaging, or an administration unit, comprising: a packaging material; the above defined combination, pharmaceutical composition or pharmaceutical kit; and a label or package insert contained within said packaging material, indicating that said combination, pharmaceutical composition or pharmaceutical kit is administered to a patient for the treatment of cancer.

Further non-limiting exemplary embodiments of the present disclosure are shown below.
1. A method of treating metastatic or advanced breast cancer comprising administering to a patient in need thereof, amcenestrant at a dose of 200 mg per day in combination with palbociclib or a pharmaceutically acceptable salt thereof.
2. A method of treating metastatic or advanced breast cancer comprising administering to a patient in need thereof amcenestrant at a dose of 200 mg per day, wherein said patient is also on palbociclib therapy.
3. A method of treating metastatic or advanced breast cancer comprising administering to a patient in need thereof, amcenestrant at a dose of 200 mg per day and palbociclib at a dose of 125 mg per day.
4. The method according to embodiment 1 or embodiment 2, wherein palbociclib is at a dose of 125 mg per day.
5. The method according to any of embodiments 1 to 4, wherein said method is for a human patient.
6. The method according to any of embodiments 1 to 4, wherein the breast cancer is an estrogen receptor positive cancer.
7. The method according to any of embodiments 1 to 4, wherein the breast cancer is a human epidermal growth factor receptor 2 negative cancer.
8. The method according to any of embodiments 1 to 4, wherein the breast cancer is an estrogen receptor positive, human epidermal growth factor receptor 2 negative cancer.
9. The method according to any of embodiments 1 to 8, wherein the patient is a woman.
10. The method according to any of embodiments 1 to 9, wherein the patient is a postmenopausal woman.
11. Amcenestrant or a pharmaceutically acceptable salt thereof, for use in the treatment of metastatic or advanced breast cancer at a dose of 200 mg per day, in combination with palbociclib or a pharmaceutically acceptable salt thereof.
12. The compound for use according to embodiment 11, wherein palbociclib is at a dose of 125 mg per day.
13. The compound for use according to embodiment 11 or 12, wherein said use is for a human patient.
14. The compound for use according to any of embodiments 11 to 13 wherein the breast cancer is an estrogen receptor positive cancer.
15. The compound for use according to any of embodiments 11 to 13, wherein the breast cancer is a human epidermal growth factor receptor 2 negative cancer.
16. The compound for use according to any of embodiments 11 to 13, wherein the breast cancer is an estrogen receptor positive, human epidermal growth factor receptor 2 negative cancer.
17. The compound for use according to any of embodiments 11 to 16, wherein the patient is a woman.
18. The compound for use according to any of embodiments 11 to 16, wherein the patient is a postmenopausal woman.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of neurology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

In order that this invention may be better understood, the following examples are set forth. These examples are for purposes of illustration only and are not to be construed as limiting the scope of the invention in any manner.

### List of Abbreviations

aBC: advanced breast cancer
AE: adverse event
AI: aromatase inhibitor
ALT: alanine aminotransferase
AST: aspartate aminotransferase
AUC: Area under the plasma concentration-time curve
BID: twice daily
BOR: best overall response
CBR: clinical benefit rate
CDK: cyclin dependent kinase
CDK4/6i: cyclin dependent kinase 4/6 inhibitor
cfDNA: circulating free DNA
COD: cut-off date
CR: complete response
DCR: disease control rate
ddPCR: digital droplet polymerase chain reaction
DLT: dose limiting toxicity
ECOG: Eastern Cooperative Oncology Group
ER: estrogen receptor
ER+: estrogen receptor positive
ESR1: estrogen receptor 1
ET: endocrine therapy
FES-PET: Fluoroestradiol Positron-emission tomography
HER2: human epidermal growth factor receptor 2
HER2−: human epidermal growth factor receptor 2 negative
hr: hour
IB: Investigator Brochure
MAD: Maximum Administered Dose
MedDRA PT: Medical Dictionary for Regulatory Activities, Preferred Terms
mTORi: mammalian target of rapamycin inhibitor
NCI-CTCAE: National Cancer Institute Common Terminology Criteria for Adverse Events
ORR: objective response rate
PET: positron emission tomography
PI3K: phosphoinositide 3-kinase
PD: progressive disease
PDy: pharmacodynamic(s)
PFS: Progression-Free Survival
PgR: progesterone receptor
PgR+: progesterone receptor positive
PK: pharmacokinetic(s)
PR: partial response
QD: once daily
RD: recommended dose
RDI relative dose intensity
RECIST: Response Evaluation Criteria in Solid Tumors
RP2D: Recommended Phase 2 Dose
SAE: serious adverse event
SD: stable disease
SERD: Selective estrogen receptor degrader
SERM: selective estrogen receptor modulator
SOC: system organ class
TEAE: treatment-emergent adverse event
TRAE: treatment-related adverse event EXAMPLES

### Example 1: Clinical Trial Protocol

This Example describes the clinical trial protocol used for the study described in the Examples below. This study is a Phase 1/2 study for the safety, efficacy, pharmacokinetic and pharmacodynamics evaluation of amcenestrant, administered orally in combination with palbociclib in postmenopausal women with estrogen receptor-positive advanced breast cancer.

Study Objectives

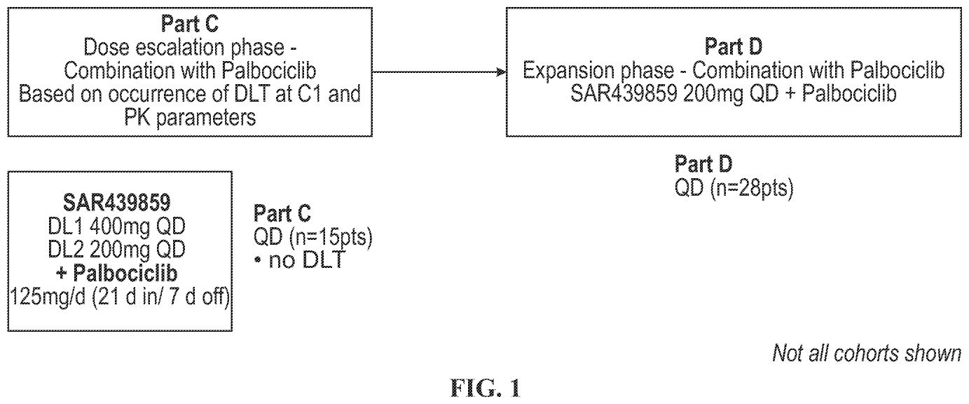

For the escalation combination study with palbociclib (“Part C”), the primary objective of the study is to assess the incidence rate of dose-limiting toxicity (DLT) and to determine the maximum tolerated dose (MTD) as well as the recommended dose (RD) of amcenestrant administered in combination with palbociclib, in postmenopausal women with estrogen receptor (ER) positive and human epidermal growth factor receptor 2 (HER2) negative advanced breast cancer (FIG. 1).

For the dose expansion study of the combination therapy with amcenestrant and palbociclib (“Part D”), the primary objective of the study is to characterize the overall safety profile of amcenestrant administered in combination with palbociclib (FIG. 1).

The secondary objectives of the Part C and Part D (unless mentioned otherwise) are:
to characterize the overall safety profile of amcenestrant administered in combination with palbociclib (Part C);
to characterize the pharmacokinetic (PK) profile of amcenestrant administered in combination with palbociclib, as well as the PK profile of palbociclib in combination with amcenestrant;
to evaluate the antitumor activity using ORR according to RECIST v1.1 of amcenestrant administered in combination with palbociclib, the clinical benefit rate (CBR, complete response (CR), partial response (PR) and stable disease (SD) after ≥24 weeks), and progression-free survival (PFS);
to evaluate the ORR and CBR (CR, PR and SD after ≥24 weeks) in Part D according to the estrogen receptor 1 (ESR1) gene mutational status (mutant and wild type) at baseline and during treatment; and
to evaluate the time to first tumor response (CR or PR) in Part D.

The exploratory objectives are to evaluate the PK/pharmacodynamic (PD) relationships of amcenestrant and palbociclib and to determine the mutational profiles of tumor genes over time (i.e., change between baseline and end of treatment (EOT)) by circulating free DNA (cfDNA) analysis.

Study Design

This is an open-label, non-comparative, dose escalation and dose expansion, safety, efficacy, PK and PD evaluation study of amcenestrant administered orally in combination with palbociclib in postmenopausal women with ER positive and HER2 negative advanced breast cancer. Part C is a dose escalation study to evaluate the safety, PK and PD of amcenestrant administered in combination with the recommended standard dosage of palbociclib. Part D is a dose expansion study to characterize the overall safety, PK and PD of amcenestrant administered at the selected dose(s) (from Part C) in combination with palbociclib.

Part C is expected to assess two amcenestrant dose levels schedule using the 3+3 standard dose escalation design with palbociclib given at fixed dose. An additional BID dose could be tested, if needed. The dose levels of the two drugs are shown in the table below.

TABLE 5
Amcenestrant dose levels in Part C

| Dose levels (DL)^{a} | Amcenestrant | Palbociclib ^{b} |
|---|---|---|
| DL1 QD | 200 mg | 125 mg |
| DL2 QD | 400 mg | 125 mg |
| DL3 BID | 400 mg | 125 mg |

^{a}Lower dose, intermediate dose levels and a BID dose regimen can also be tested.
^{b} Oral route once daily with food for 21 days followed by 7 days off therapy to comprise a complete cycle of 28 days. A lower dose (e.g., 100 mg, 75 mg) can be proposed depending on tolerance.

Three to six evaluable patients will be treated at each dose level, and dose escalation decision will be based on DLT observed for at least one cycle duration (i.e., 28 days) of the first three evaluable patients. If one of the first three evaluable patients experiences DLT during Cycle 1, this cohort will be expanded with a total of six patients. If none of the first three patients or less than two out of six patients experienced a DLT, the dose escalation will proceed to the next dose level. The second and third patients of a given cohort can only be enrolled when the first patient will have received one week of amcenestrant and palbociclib without DLT. The enrollment at the next dose level may not proceed before at least 3 patients treated at the current dose level have been followed for at least one cycle duration (i.e., 28 days) and are evaluable for DLT assessment.

Patients who discontinue study treatment prematurely before the end of DLT observation period for any reason other than DLT must be replaced.

As a rule, the dose escalation will stop when the MAD, the dose at which ≥33% (2 patients out of up to 6) of evaluable patients have experienced a DLT at Cycle 1, is reached. The MTD is defined as the highest dose level at which no more than 1 patient of a maximum of 6 evaluable patients experienced a DLT. The MTD is one dose level below the MAD or the highest dose tested if MAD is not reached.

Although the dose escalation process is guided by the safety evaluation during Cycle 1 of treatment, cumulative or irreversible toxicities observed after subsequent administrations will also be considered for the dose escalation and the dose selection decisions (i.e., expansion of a given dose level, intermediate dose levels), as well as any relevant information, upon recommendation from the study committee.

For Part D of the study, when the dose escalation phase (Part C) ends, at least one RD will be proposed for the expansion cohort (Part D) and approximately 28 patients will be treated at each selected RD (from Part C).

Study Population

The inclusion criteria for the two Parts of the study are as follows.

I 01. Postmenopausal women as defined by one of the following:
a) women >60 years; or
b) women ≤60 years:
   with spontaneous cessation of menses >12 months prior to registration in the absence of chemotherapy, tamoxifen and toremifene;
   or with cessation of menses of duration ≤12 months or secondary to hysterectomy and have follicle stimulating hormone (FSH) level in the postmenopausal range according to institutional standards (or >34.4 IU/L if institutional range is not available) prior to registration;
   or who have received hormonal replacement therapy but who have discontinued this treatment and have FSH level in the postmenopausal range according to institutional standards (or >34.4 IU/L if institutional range is not available) prior to registration;
   or with status post bilateral surgical oophorectomy;
   or are premenopausal women on a gonadotropin-releasing hormone (GnRH) analog for at least 6 months (to be continued during study treatment) and have a negative pregnancy test prior to initiation of study treatment and at monthly intervals during treatment.

I 02. Patients with histological or cytological proven diagnosis of the breast adenocarcinoma with evidence of either locally advanced not amenable to radiation therapy or surgery in a curative intent, inoperable and/or metastatic disease.

I 03. Either the primary tumor or any metastatic site is positive for ER (>1% tumor cell staining by immunohistochemistry (IHC).

I 04. Either the primary tumor or any metastatic site is HER2 non-overexpressing by IHC (0, 1+) or in situ hybridization-negative based on single-probe average HER2 copy number <4.0 signals/cell or dual-probe HER2/centromeric probe for chromosome 17 (CEP17) ratio <2 with an average HER2 copy number <4.0 signals/cell as per the American Society of Clinical Oncology guidelines.

I 05. Prior chemotherapy for advanced/metastatic disease is allowed (including antibody drug conjugates, considered as chemotherapy in the study), but patients must have received no more than 1 prior chemotherapeutic regimen.

I 06. Included patients must have received at least 6 months of prior endocrine therapy for advanced breast cancer. For Part D, no more than 2 prior lines of endocrine therapy are allowed. Patients with early progression on adjuvant endocrine therapy or who progressed on adjuvant endocrine therapy within 12 months after completion are eligible.

I 07. All included patients are ≥18 years old.

I 08. Patients have measurable lesion by RECIST v1.1.

The exclusion criteria for patient selection include the following.

Exclusion Criteria Related to Study Methodology:

E 01. Eastern Cooperative Oncology Group (ECOG) performance status ≥2.

E 02. Significant concomitant illness, including psychiatric condition that, in the opinion of the investigator or sponsor, would adversely affect the patient's participation in the study.

E 03. Medical history or ongoing gastrointestinal disorders potentially affecting the absorption of amcenestrant and/or palbociclib.

E 04. Any malignancy related to human immunodeficiency virus (HIV) or unresolved viral hepatitis.

E 05. Patients with a life expectancy less than three months.

E 06. Patient not suitable for participation, whatever the reason, as judged by the investigator, including medical or clinical conditions, or patients potentially at risk of noncompliance to the study procedures.

E 07. Major surgery within four weeks prior to the first study treatment administration.

E 08. Patient with any other cancer. However, adequately treated basal cell or squamous cell skin cancer or in situ cervical cancer or any other cancer from which the patient has been disease free for >3 years are allowed.

Exclusion Criteria Related to the Disease:

E 11. Patients with known brain metastases, leptomeningeal carcinomatosis or/and spinal cord compression; patients with brain metastases that have been previously totally resected or irradiated are eligible provided no progression or relapse is observed within four weeks of the treatment.

E 12. Treatment with anticancer agents (including investigational drugs) less than 2 weeks before first study treatment administration (less than four weeks if the anticancer agents were antibodies).

E 13. Prior treatment with another selective ER down-regulator (SERD) except fulvestrant for which a washout of at least six weeks is required prior to the first study drug administration.

E 14. Inadequate hematological function including neutrophils <1.5×10⁹/L; platelet count <100×10⁹/L.

E15. Prothrombin time: International normalized ratio >1.5 times the upper limit of normal (ULN) or out of therapeutic range if receiving anticoagulation that would affect the PT/INR.

E 16. Inadequate renal function with serum creatinine ≥1.5×ULN or, if between 1.0 and 1.5×ULN with eGFR <60 mL/min/1.73 m² as estimated using the abbreviated Modification of Diet in Renal Disease formula.

E 17. Liver function: aspartate aminotransferase (AST) >3×ULN, or alanine aminotransferase (ALT) >3×ULN. Alkaline phosphatase (ALP) up to Grade 2 (2.5 to 5×ULN), gamma glutamyl transferase (GGT) up to Grade 2 (2.5 to 5×ULN) would be acceptable only if related to the presence of bone and/or liver metastases. Total bilirubin >1.5×ULN).

E 18. Patients with Gilbert disease.

E 19. Non-resolution of any prior treatment related toxicity to <Grade 2, except for alopecia according to National Cancer Institute Common Terminology Criteria for adverse events (NCI-CTCAE) v4.03.

E 20. Treatment with atazanavir, lopinavir (antiviral agents), probenecid, ketoconazole (antifungal) and quercetin (antioxidant) and treatment that are P-gP substrate (dabigatran, digoxin, fexofenadine), less than two weeks before first study treatment administration or 5 elimination half-lives, whichever is longest.

E 21. Treatment with strong and moderate CYP3A or CYP2C₈ inducers within two weeks before first study treatment administration or five elimination half-lives, whichever is longest.

E 22. Up to one prior CDK4/6 inhibitor based therapy is allowed. No prior CDK4/6 exposure is required for patients with early progression on adjuvant endocrine therapy or who progressed on adjuvant endocrine therapy within 12 months after completion of adjuvant endocrine therapy.

E 23. Treatment with strong CYP3A inhibitors within two weeks before first study treatment administration or five elimination half-lives, whichever is longest.

E 24. Medical conditions requiring concomitant administration of medications with a narrow therapeutic window metabolized by CYP3A and for which a dose reduction cannot be considered.

E 25. For Part D only, the following prior therapies for advanced disease are not allowed: CDK4/6 inhibitors, PI3K inhibitors and mTOR inhibitors.

Dosing Regimen and Formulation

Patients will take 100 mg capsules of amcenestrant by oral route. Amcenestrant will be administered from Day 1 (without omission on Day 2) at assigned dose levels, within a 28-day cycle in fed or fasting condition, and at approximately the same time each day (±3 hours). An additional BID dose could be tested in Part C assuming that BID monotherapy indicates a benefit compared to the RD given QD.

Palbociclib (Ibrance®) is provided in 125 mg, 100 mg and 75 mg capsules and administered to the patients by oral route. The RD of palbociclib is a 125 mg capsule taken once daily for 21 consecutive days followed by 7 days off treatment (no palbociclib) to comprise a complete cycle of 28 days. Palbociclib capsules should be taken with food at approximately the same time each day. Amcenestrant and palbociclib could be taken together, i.e., with food, 5 minutes apart, regardless of the order of intake. Drugs will be taken together on Day 1 and Day 21 of Cycle 1 with full PK assessment. On other days, amcenestrant can be taken with or without food, but palbociclib is taken with food.

Clinical Endpoints

Primary Endpoint for Part C

The primary endpoint for Part C of the study is to determine the incidence of study treatment-related DLTs at Cycle 1 (Day 1 to Day 28). DLT will be defined as the occurrence of any of the following treatment-emergent adverse events (TEAEs) related to the study therapy using NCI-CTCAE (v4.03):
any Grade ≥3 nonhematological toxicity (except Grade 3 nausea and vomiting resolving to Grade ≤1 within 48 hours, with or without adequate antiemetic treatment; and Grade 3 diarrhea, if controlled with adequate antidiarrheal therapy and lasting less than 48 hours);
any Grade ≥3 hematological toxicity (except Grade 3 anemia; Grade 4 neutropenia <7 days; Grade 3 neutropenia without fever or infection; and Grade 3 thrombocytopenia without bleeding);
any elevated total serum bilirubin >2×ULN;
any toxicity related to study treatment, resulting in omission of the study treatment for 7 days or more during Cycle 1, or in Cycle 2 delay of more than 2 weeks in Part C; and
a TEAE that is possibly or probably study treatment-related and is of potential clinical significance such that further dose escalation would expose patients to unacceptable risk.
   These TEAEs will be considered as study treatment-related in the absence of evidence to the contrary and if not related to disease progression. If multiple toxicities are seen, the presence of DLTs will be based on the most severe experienced toxicity.

At the end of Cycle 1, each patient must be assessed by the Investigator as to whether or not the patient experienced DLTs, and this information must be recorded within the appropriate screen of the electronic case report forms (eCRFs) and an electronic DLT notification (either DLT or not) will be sent to the Sponsor.

Patients could continue the treatment after resolution (≤Grade 1) of the adverse event (AE) or to their baseline status.

Primary Endpoint for Part D

The primary endpoint for Part D of the study is to evaluate safety and tolerability such as type, frequency, severity, relationship to study therapy and seriousness of adverse events (AE) or laboratory abnormalities according to NCI-CTCAE v4.03.

Secondary Endpoints

The secondary endpoints, for both Parts C and D unless mentioned otherwise, are as follows:
Part C: Overall safety profile of amcenestrant, and characterization in terms of the type, frequency, severity, and relationship to study therapy of any AEs or abnormalities of physical findings, laboratory tests, or electrocardiograms (ECGs); drug discontinuation/omission/reduction and cycle delay due to AEs; or serious adverse events (SAEs).
ORR as per RECIST v1.1 assessed by investigators/local radiologists.
Part D: ORR and CBR in patients based on their ESR1 status (mutated or wild type) analyzed by multiplex droplet digital polymerase chain reaction (ddPCR) after extraction of plasma circulating DNA).
Clinical benefit (CR+PR+SD≥24 weeks) as per RECIST v1.1 assessed by investigators/local radiologists.
Duration of response assessed by investigators/local radiologists.
Progression-Free Survival (PFS), defined as the time interval from the date of the first investigational medicinal product intake to the date of the first tumor progression assessed by investigators/local radiologists per RECIST v1.1, or death (due to any cause), whichever comes first.
Part D: Time to first tumor response (CR or PR) will be defined as the time interval from the date of first administration of the study treatment to the date of the first occurrence of CR or PR, assessed by investigators/local radiologists.
PK parameters for amcenestrant during Cycle 1:
after a single dose on Day 1: at least t_{lag}, tₘₐₓ, Cₘₐₓ, AUC₀₋₂₄, or AUC₀₋₁₂;
after repeated administration on Day 22: at least tₘₐₓ, Cₘₐₓ, AUC₀₋₂₄ or AUC₀₋₁₂; and
C_{trough} (T=0 h i.e., before daily administration) will be also obtained over Cycle 1.
PK parameters for palbociclib: after first dose (Day 1) and after repeated once daily administrations (Day 22) during Cycle 1; at least tₘₐₓ, Cₘₐₓ, and AUC₀₋₂₄.

Exploratory Endpoints

An exploratory endpoint is to evaluate the ESR1 mutation status of the patients. Twelve independent mutations of ESR1 gene will be determined in all patients at baseline and at end of Cycle 2 (Day 15 to Day 28) by ddPCR from plasma extracted cfDNA.

Another exploratory endpoint is to evaluate the mutational profile of the patients. In all patients, plasma will be collected at baseline and at the EOT and cfDNA will be extracted. The mutation status of a limited number of cancer genes will be determined by next generation sequencing and the potential link between specific mutation and clinical outcomes will be investigated to understand intrinsic or acquired resistance to the amcenestrant treatment.

Yet another exploratory endpoint is to evaluate PK/PD relationships of amcenestrant with ER occupancy, PD and/or efficacy endpoints and/or other breast cancer biomarkers such as Ki67, Bcl-2 and PgR.

The clinical trial results which are described hereunder in Examples 2, 3, and 4 stem from a patient database extraction with a cut-off date of 30 Mar. 2021. Examples 5 and 6 present updated results from later patient database extractions, dated May 30, 2021 and Oct. 4, 2021, respectively. Indeed, results from the clinical trial described herein as per the protocol of Example 1 were accrued progressively, with stepwise patient database extractions at successive cut-off dates to closely monitor study evolvement.

### Example 2: Phase 1/2 Clinical Trial Results of Part C

Throughout this and the following Examples, a “cycle” of treatment refers to a four-week period in which patients received three weeks of treatment with palbociclib, followed by one week of no treatment with palbociclib, while amcenestrant is administered daily throughout the entire four-week period. Unless otherwise noted, amcenestrant and palbociclib were administered at 8:00 AM on days of medication (defined as 0 hr relative nominal time), and were taken together five minutes apart with food. On days in which palbociclib was not administered, amcenestrant was taken with or without food. Amcenestrant was orally administered as capsules containing 100 mg of amcenestrant, and palbociclib was orally administered as a capsule containing 125 mg of palbociclib.

Study treatment was continued for each patient until precluded by unacceptable toxicity, disease progression, or upon patient's request. If palbociclib was prematurely permanently discontinued, amcenestrant treatment was continued until the end of treatment (EOT) criterion was met. The EOT visit in this case was 30 days after the last amcenestrant administration date. Patients who discontinued treatment prior to documentation of PD were followed every two months until disease progression, initiation of further anti-cancer therapy, or the cut-off date (COD), whichever comes first.

Patient Population

In a dose-escalation study of amcenestrant combined with palbociclib (Part C as described in Example 1), a total of 15 patients were treated. Two dose levels of amcenestrant, 200 mg and 400 mg QD, were combined with palbociclib at a dose of 125 mg (QD for 21 consecutive days followed by 7 days off treatment—3w/1w). Nine patients were treated with amcenestrant at a dose of 200 mg and six patients were treated at a dose of 400 mg.

In Part C (baseline data), the mean age was 62.9 (9.7) years in the population (N=15), with 62.0 (10.9) and 64.2 (8.4) years in the amcenestrant 200 mg and amcenestrant 400 mg, combined with palbociclib, respectively. Eastern Cooperative Oncology Group (ECOG) status was 1 in 33.3% of the patients, and ECOG 0 for the remaining patients. The median time from first diagnosis to first study treatment administration was 7.33 (4.53) years.

All patients were ER positive and HER2 negative, and 66.7% of the patients were progesterone receptor positive (PgR).

All patients had metastatic disease, except for one patient who had locally advanced disease in the amcenestrant 200 mg combined dose sub-group. A median of three organs involved at study entry (range 1 to 5) was observed; the main organs involved were lymph nodes and bone (60.0% each), lung (46.7%) and liver (40%).

All patients except one (N=14; 93.3%) received at least one prior anti-cancer therapy for advanced disease (range 0-4) among whom 7 (50.0%) patients received a prior chemotherapy. A total of ten (66.7%) patients received a prior targeted therapy: among them, 50.0% received prior CDK4/6 inhibitor and 40.0% patients had prior mTOR inhibitor. All patients except one (N=14; 93.3%) received a prior hormonotherapy: among them, 85.7% received prior aromatase inhibitors (AI), 28.6% received prior SERD-based therapy and 35.7% prior SERM.

Exposure

A total of 15 patients were treated in the dose escalation of amcenestrant in combination with palbociclib (Part C). At the cut-off date of this Example, 4 (26.7%) patients remained on study treatment. The median duration of treatment of amcenestrant was 24.3 weeks (range 8-101 weeks), and palbociclib was 24.0 weeks (range 8-101 weeks). The median relative dose intensity (RDI) was 98.1% and 96.8% for amcenestrant and palbociclib, respectively. RDI is defined as the ratio of the actual dose intensity to the planned dose intensity, and is an indicator of the feasibility of the chosen schedule of administration. A total of 53.3% of patients received ≥5 cycles of study treatment. Two patients (13.3%) had at least one dose reduction of amcenestrant and five patients (33.3%) had at least one dose reduction of palbociclib. Five patients (33.3%) had at least 7 consecutive days of dose omission. One patient (6.7%) discontinued the study treatment due to a non-serious adverse event, asthenia, considered related to both 200 mg amcenestrant and palbociclib treatment.

Safety

At the COD, 9 patients received amcenestrant 200 mg QD in combination with palbociclib, and 6 patients received amcenestrant 400 mg QD in combination with palbociclib 125 mg. All patients completed dose limiting toxicity (DLT) observation period; no DLTs were reported in either dose level tested of amcenestrant in combination with palbociclib.

The safety profile among the 15 patients in Part C of the study (safety population, Part C) is presented below in Table 6 (TEAE: Treatment Emergent Adverse Event; SAE: Serious Adverse Event).

TABLE 6
Treatment Emergent Adverse Events (TEAE) by Dose Level

| | Amcenestrant + Palbociclib (125 mg) | | | | | |
|---|---|---|---|---|---|---|
| | 200 mg | | 400 mg | | All | |
| n (%)* | (N = 9) | | (N = 6) | | (N = 15) | |
| Participants with any TEAE | 9 | (100) | 6 | (100) | 15 | (100) |
| Participants with any grade ≥3 TEAE | 6 | (66.7) | 1 | (16.7) | 7 | (46.7) |
| Participants with any grade 3-4 TEAE | 6 | (66.7) | 1 | (16.7) | 7 | (46.7) |
| Participants with any grade 5 TEAE | 0 | | 0 | | 0 | |
| occurring during the treatment period | | | | | | |
| Participants with any treatment emergent | 2 | (22.2) | 1 | (16.7) | 3 | (20.0) |
| SAE | | | | | | |
| Participants with any TEAE related to | 9 | (100) | 6 | (100) | 15 | (100) |
| Amcenestrant | | | | | | |
| Participants with any TEAE related to | 9 | (100) | 3 | (50.0) | 12 | (80.0) |
| Palbociclib | | | | | | |

| | | | | | |
|---|---|---|---|---|---|
| Participants with any TEAE leading to | 1 | (11.1) | 0 | 1 | (6.7) |
| definitive treatment discontinuation | | | | | |

| | | | |
|---|---|---|---|
| Participants with any TEAE leading to | 0 | 0 | 0 |
| premature discontinuation of palbociclib | | | |

*n(%) = number and percentage of participants with at least one TEAE.

All 15 patients (100%) experienced at least one TEAE. TEAEs Grade 3 were reported in seven (46.7%) of patients. The most frequently reported non-hematological TEAEs by MedDRA PT, regardless of the relationship to amcenestrant or palbociclib, were nausea (33.3%) in 5 patients, diarrhea, and vomiting, pain in extremity, abdominal pain, upper abdominal pain, and asthenia in four patients each (26.7% each); cough, fatigue, non-cardiac chest pain, arthralgia, muscle spasm, vertigo, and hot flush in three patients each (20.0% each); urinary tract infection, dizziness, constipation, alopecia, dry skin, malaise, influenza like illness, decreased appetite, eye edema, pulmonary embolism and pruritus in two patients each (13.3% each).

TEAEs with Grade ≥3 were reported in a total of seven (46.7%) patients, six of which received amcenestrant at a dose of 200 mg QD, and one of which received amcenestrant at a dose of 400 mg QD. Non-hematological TEAEs Grade ≥3, regardless of the relationship to amcenestrant or palbociclib, were gastritis, febrile neutropenia, pulmonary embolism, small intestine obstruction, and alanine transaminase (ALT) increase in one patient each (6.7% each).

All patients experienced TEAEs considered to be related to amcenestrant by the Investigator. The most frequently non-hematological reported amcenestrant-related TEAEs by MedDRA PT occurring in at least three patients, were asthenia in four patients (26.7%); diarrhea, hot flush, fatigue and nausea in three patients each (20.0% each). Non-hematological Grade ≥3 events related to amcenestrant have been reported in one patient (6.7%): febrile neutropenia.

A total of 12 patients (80.0%) experienced TEAEs considered to be related to palbociclib. The most frequently reported palbociclib-related non-hematological TEAEs by MedDRA PT occurring in at least three patients were asthenia in four patients (26.7%); nausea and fatigue in three patients each (20.0% each). Non-hematological Grade ≥3 events related to palbociclib have been reported in two patients (13.3%), febrile neutropenia and ALT increase.

In general, no dose dependency was observed in the TEAE profile (regardless of the patient's dose of amcenestrant or palbociclib).

Four serious TEAEs were reported in three patients (20.0%). All serious TEAEs were considered as not related to either amcenestrant or palbociclib. At a dosage of 200 mg amcenestrant with palbociclib, one patient presented gastritis and small intestinal obstruction, and another patient presented Coronavirus infection. At a dosage of 400 mg amcenestrant with palbociclib, abdominal pain was reported in one patient. No patients died during the treatment period.

Further details of the non-hematological amcenestrant treatment-related adverse events (TRAEs) and non-hematological palbociclib TRAEs, respectively, are provided in Table 7 and Table 8 below, summarizing those events for all of Part C, Part D of the study and pooled Parts C+D safety population (“RP2D”: recommended Phase 2 dose).

TABLE 7
Non-hematological amcenestrant TRAEs occurring in ≥3 patients in the safety
population treated with amcenestrant at RP2D + palbociclib

| | Dose Escalation | | Dose Expansion | | Safety Population | |
|---|---|---|---|---|---|---|
| | Amcenestrant 200 or | | Amcenestrant 200 mg + | | Amcenestrant 200 mg + | |
| | 400 mg + Palbociclib | | Palbociclib | | Palbociclib | |
| Amcenestrant | (Part C; N = 15) | | (Part D; N = 30) | | (Parts C + D; N = 39) | |
| TRAEs, n (%) | All Grades | Grade ≥3 | All Grades | Grade ≥3 | All Grades | Grade ≥3 |
| Nausea | 3 (20.0) | 0 | 4 (13.3) | 0 | 7 (17.9) | 0 |
| Fatigue | 3 (20.0) | 0 | 5 (16.7) | 0 | 7 (17.9) | 0 |
| Arthralgia | 1 (6.7) | 0 | 3 (10.0) | 0 | 4 (10.3) | 0 |
| Asthenia | 4 (26.7) | 0 | 1 (3.3) | 0 | 4 (10.3) | 0 |
| Hot flush | 3 (20.0) | 0 | 1 (3.3) | 0 | 4 (10.3) | 0 |
| Diarrhea | 3 (20.0) | 0 | 2 (6.7) | 0 | 3 (7.7) | 0 |
| Dysgeusia | 1 (6.7) | 0 | 2 (6.7) | 0 | 3 (7.7) | 0 |
| Dry skin | 1 (6.7) | 0 | 2 (6.7) | 0 | 3 (7.7) | 0 |
| GERD | 1 (6.7) | 0 | 2 (6.7) | 0 | 3 (7.7) | 0 |
| (gastroesophageal | | | | | | |
| reflux disease) | | | | | | |

TABLE 8
Non-hematological palbociclib TRAEs occurring in ≥3 patients in the safety
population treated with amcenestrant at RP2D + palbociclib

| | Dose Escalation | | Dose Expansion | | Safety Population | |
|---|---|---|---|---|---|---|
| | Amcenestrant 200 or | | Amcenestrant 200 mg + | | Amcenestrant 200 mg + | |
| | 400 mg + Palbociclib | | Palbociclib | | Palbociclib | |
| Palbociclib | (Part C; N = 15) | | (Part D; N = 30) | | (Parts C + D; N = 39) | |
| TRAEs, n (%) | All Grades | Grade ≥3 | All Grades | Grade ≥3 | All Grades | Grade ≥3 |
| Fatigue | 3 (20.0) | 0 | 10 (33.3) | 0 | 12 (30.8) | 0 |
| Nausea | 3 (20.0) | 0 | 7 (23.3) | 0 | 10 (25.6) | 0 |
| Asthenia | 4 (26.7) | 0 | 1 (3.3) | 0 | 4 (10.3) | 0 |
| Dysgeusia | 1 (6.7) | 0 | 3 (10.0) | 0 | 4 (10.3) | 0 |
| Stomatitis | 1 (6.7) | 0 | 3 (10.0) | 0 | 4 (10.3) | 0 |
| Abdominal | 1 (6.7) | 0 | 2 (6.7) | 0 | 3 (7.7) | 0 |
| pain upper | | | | | | |
| Diarrhea | 1 (6.7) | 0 | 3 (10.0) | 0 | 3 (7.7) | 0 |
| Dizziness | 1 (6.7) | 0 | 2 (6.7) | 0 | 3 (7.7) | 0 |
| Dry mouth | 1 (6.7) | 0 | 2 (6.7) | 0 | 3 (7.7) | 0 |
| Dry skin | 1 (6.7) | 0 | 2 (6.7) | 0 | 3 (7.7) | 0 |
| GERD | 1 (6.7) | 0 | 2 (6.7) | 0 | 3 (7.7) | 0 |
| (gastroesophageal | | | | | | |
| reflux disease) | | | | | | |

Regarding laboratory abnormalities for Part C of the study, no grade 3 or 4 anemia was observed. Neutrophil count decrease was observed in 13 patients (86.7%). All patients were Grade 0 at baseline during treatment period: seven patients (46.7%) increased to Grade 3; four patients (26.7%) increased to Grade 2; and two patients (13.3%) increased to Grade 1. Lymphocyte count decrease was observed in 12 patients (80%) during treatment period, three patients (20.0%) had Grade 3, seven patients (46.7%) had Grade 2, and two patients had Grade 1. No Grade 3 AST were observed. One patient (6.7%) demonstrated an ALT measurement changing from normal at baseline to Grade 3. No increase in blood bilirubin was observed in any patients.

Pharmacokinetics

The pharmacokinetics (PK) of both amcenestrant and palbociclib were also assessed during Part C of the study, with amcenestrant (200 or 400 mg continuous dosing) in combination with palbociclib (125 mg QD for 21 consecutive days followed by seven days off treatment—3w/1w). A summary of exposure parameters of amcenestrant obtained at steady state after 21 days of continuous dosing in combination with palbociclib is provided in Table 9. For reference, the PK results of amcenestrant used as a monotherapy at a dose of 400 mg are also provided in the table. Summary statistics are median (min-max) for tₘₐₓ, geometric mean [90% confidence Interval] for Rac (accumulation ratio), and arithmetic mean (CV %) [geometric mean] for other PK parameters.

TABLE 9
Amcenestrant PK Parameters at Steady State

| Amcenestrant | Tₘₐₓ | Cₘₐₓ | AUC₀₋₂₄ₕ | C_{trough} | CLss/F | Rac, AUC₀₋₂₄ |
|---|---|---|---|---|---|---|
| Dose group | (h) | (ng/mL) | (ng · h/mL) | (ng/mL) | (L/h) | (Day 21/Day 1) |
| 200 mg | 4.88 | 2060 | 20 600 | 226 | 10.7 | 1.02 (N = 7) |
| with | (1.37 -8.17) | (40%) | (36%) | (38%) | (32%) | (0.54-1.63) |
| palbociclib | | [1940] | [19500] | [215] | [10.2] | |
| (N = 8) | | | | | | |
| 400 mg | 4.00 | 4350 | 33 100 | 280 | 13.5 | 0.69 |
| with | (1.83-10) | (73%) | (35%) | (48%) | (40%) | (0.38-1.15) |
| palbociclib | | [3450] | [31400] | [248] | [12.7] | |
| (N = 5) | | | | | | |
| 400 mg | 3.00 | 4380 | 43 200 | 630 | 10.6 | 0.93 |
| monotherapy | (1.02-6.00) | (28%) | (38%) | (87%) | (41%) | (0.49-1.90) |
| (N = 13) | | [4220] | [40400] | [466] | [9.90] | |

Treatment with palbociclib 125 mg QD 3w/1w did not show a major effect on the PK of amcenestrant. The systemic clearance of amcenestrant on Day 21 ranged from between 10.7±3.38 L/h at 200 mg and 13.5±5.36 L/h at 400 mg, which is similar or slightly higher (28%) to the systemic clearance observed after repeated administration of amcenestrant at 400 mg QD as single agent, regardless of food status (10.6±4.35 L/h).

A summary of palbociclib PK parameters obtained at steady state after administrations in fed conditions in combination with amcenestrant is presented in Table 10. Also provided for reference are published exposure data describing the pharmacokinetic parameters of palbociclib when given as a monotherapy or in combination with letrozole. Summary statistics of palbociclib historical data show geometric mean (CV %) except for A5481001 study with arithmetic mean. For the present trial (“This Study”), summary statistics show arithmetic mean (CV %) ([geometric mean]).

TABLE 10
Palbociclib PK Parameters at Steady State

| | | Cₘₐₓ | AUC_{0-24 h} | % AUC |
|---|---|---|---|---|
| Study | Therapy type | (ng/mL) | (ng · h/mL) | decrease ¹ |
| A5481001 (N = 4) | palbociclib | 97.4 (41%) | 1 733 (42%) | — |
| | monotherapy | | | |
| A5481003 (N = 12) | palbociclib | 116 (28%) | 1 982 (29%) | — |
| | monotherapy | | | |
| A5481008 (N = 41) | palbociclib with | 110 (35%) | 1 992 (35%) | — |
| | letrozole | | | |
| Reference - | | 110 | 1 972 | Reference |
| Geometric mean of | | | | value |
| individual studies | | | | |
| above, weighed by | | | | |
| number of subjects | | | | |
| (N = 57) | | | | |
| This Study (N = 8) | palbociclib with | 98.1 (38%) | 1 660 (45%) | 23% |
| | 200 mg amcenestrant | [91.8] | [1520] | |
| This Study (N = 5) | palbociclib with | 59.4 (47%) | 823 (32%) | 60% |
| | 400 mg amcenestrant | [53.0] | [782] | |

¹ The % AUC decrease is calculated as geometric mean of [AUC_{0-24 h} palbociclib (ref) − palbociclib + amcenestrant]/AUC_{0-24 h} of palbociclib (ref) × 100.
Palbociclib trials:
A5481001: Phase 1 dose escalation in solid tumor (Flaherty et al., Clin Cancer Res. (2012) 18(2): 568-76).
A5481003: PALOMA-1, Phase 1 clinical trial (Ibrance ® Health Canada Product Monograph, Submission Control No: 234582 (2020)).
A5481008: PALOMA-2, Phase 3 clinical trial (Durairaj et al., Anti-Cancer Drugs. (2018) 29(3): 271-80).

The above data show that, when combined with increasing doses of amcenestrant, palbociclib at 125 mg QD 3w/1w had decreased exposure in patients. Preliminary analysis showed an average decrease of palbociclib exposure by 23% when combined with 200 mg amcenestrant QD (N=8) and by 57% when combined with 400 mg amcenestrant QD (N=3), using published palbociclib data as reference. With the data of two additional patients at 400 mg amcenestrant QD (N=5), the decrease was 60%.

The extent of decrease in palbociclib exposure in patients treated with 200 mg amcenestrant was not considered as clinically relevant according to the palbociclib labelling. Palbociclib clearance is mainly metabolic and showed a 32% decrease of exposure when combined with a moderate CYP3A inducer (modafinil), according to Ibrance®'s U.S. label. Accordingly, the effect of amcenestrant 200 mg QD on palbociclib 125 mg QD 3w/1w is not considered as clinically relevant.

Antitumor Activity

In Part C, all 15 patients were evaluable as per RECISTv.1.1 (Table 11). Two patients (13.3%), both treated with 200 mg QD amcenestrant, had confirmed PR. Seven patients (46.7%) had SD, four of which were treated with 200 mg QD amcenestrant with palbociclib. Six patients (40.0%) had PD, including three patients treated with 200 mg amcenestrant with palbociclib.

Clinical benefit (CR+PR+SD at ≥24 weeks) was observed in seven patients (CBR=46.7%), including four patients treated with 200 mg amcenestrant with palbociclib. The clinical benefit was observed in 1/6 patients with ESR1 mutations (CBR=16.7%) and in 6/9 patients with wild-type ESR1 (CBR=66.7%).

In addition, tumor shrinkage was observed in nine (60.0%) patients, including five patients at 200 mg amcenestrant.

TABLE 11
Best Overall Response

| | Amcenestrant + Palbociclib (125 mg) | | |
|---|---|---|---|
| | 200 mg | 400 mg | All |
| | (N = 9) | (N = 6) | (N = 15) |
| Best Overall Response [n(%)] | | | |
| Number | 9 | 6 | 15 |
| Complete Response (CR)^{a} | 0 | 0 | 0 |
| Partial Response (PR)^{a} | 2 (22.2) | 0 | 2 (13.3) |
| Stable Disease (SD) | 4 (44.4) | 3 (50.0) | 7 (46.7) |
| Progressive Disease (PD) | 3 (33.3) | 3 (50.0) | 6 (40.0) |
| Objective Response Rate | 2 (22.2) | 0 | 2 (13.3) |
| (confirmed CR and PR) [n(%)] | | | |
| 90% CI^{b} | (4.1%, 55.0%) | NC | (2.4%, 36.3%) |
| Clinical Benefit Rate [n(%)]^{c} | 4 (44.4) | 3 (50.0) | 7 (46.7) |
| 90% CI^{b} | (16.9%, 74.9%) | (15.3%, 84.7%) | (24.4%, 70.0%) |

^{a}Confirmation of response is required (i.e., a second examination done at least 4 weeks apart [i.e., >=28 days], in order to confirm the antitumoral response)
^{b}Estimated by Clopper Pearson interval
^{c}Clinical Benefit Rate: CR, PR or prolonged SD (meaning stable disease for 24 weeks or more)

### Conclusion

Overall, the combination therapy with amcenestrant and palbociclib was well tolerated with a favorable safety profile at the two tested amcenestrant dose levels during this dose escalation part; no DLTs were observed. Adverse events observed were generally of Grade 1 or 2. The incidence of neutrophil count decrease with amcenestrant and palbociclib (86.7%) was found similar to values reported in literature on palbociclib.

However, a dose dependent effect of amcenestrant on palbociclib exposure was observed with a decrease of palbociclib Cₘₐₓ and AUC. PK data showed that palbociclib AUC was decreased by 23% in combination with 200 mg amcenestrant (N=8) and by 60% with 400 mg amcenestrant (N=5), respectively, when compared to palbociclib historical published data.

Decreasing the dose of amcenestrant from 400 mg to 200 mg is not expected to have a negative effect on the molecule's anti-tumor activity. ¹⁸F-FES PET inhibition has previously demonstrated that, when used as a monotherapy, a dose from 150 mg amcenestrant is sufficient to mediate greater than 90% target engagement. The decrease in palbociclib exposure observed when palbociclib was administered with a dose of 200 mg amcenestrant is not considered clinically relevant. Based on these findings and the favorable safety profile of combination treatment, the recommended dose of the combination treatment of 200 mg amcenestrant with 125 mg palbociclib was chosen for further studies.

### Example 3: Phase 1/2 Clinical Trial Results of Part D

This Example describes a dose expansion undertaken with patients treated with 200 mg QD amcenestrant combined with 125 mg QD 3w/1w palbociclib. A total of 30 patients were treated. All 30 patients were evaluated for safety, and 29 were evaluated for activity. The COD was when the last patient had had 8 cycles of treatment or had earlier discontinued the study.

Patient Characteristics

The mean age was 59.5 (range 33-86) years. The ECOG status was 1 in seven (23.3%) of the patients, and the remainder of the patients had an ECOG status of 0. The median time from first diagnosis to first study treatment administration was 6.52 years (with a range of 1.7 to 29.5 years). All patients were ER+/HER2−/PgR+ except for two patients who were ER+/HER2−/PgR− (7.1%). Patients had a median of two organs involved at study entry (range of one to six); the main organs involved were bone (46.7), lung and lymph nodes (43.3% each), liver (33.3%) and pleura (20.0%). A total of 15 (50.0%) patients had received at least one prior anti-cancer treatment in advanced setting, among whom four (26.7%) patients received prior chemotherapy. One (6.7%) patient received prior targeted therapy. A total of 14 (93.3%) patients had received prior hormonotherapy, among whom eleven (78.6%) received prior AI, three (21.4%) received SERM, and one (7.1%) had previously received SERD-based therapy. The remaining 15 patients (50%) previously recurred during adjuvant therapy while receiving AI or SERM therapy.

Safety

The overall safety profile of Part D is presented in Table 13. A total of 27 (90.0%) experienced at least one TEAE. TEAEs Grade ≥3 were reported in fifteen (50.0%) of patients. Further details of the non-hematological amcenestrant TRAEs and non-hematological palbociclib TRAEs, occurring in at least 3 patients, are provided in Table 7 and Table 8, respectively. Neutrophil count decrease based on hematological laboratory abnormalities was observed in the majority of patients (93.3%; 56.7% Grade ≥3).

The global safety analysis including details from all patients treated at 200 mg amcenestrant in combination with palbociclib are presented in the section “Pooled Population safety” for Parts C and D.

Antitumor Activity

An ORR was observed in 10 patients (34.5%) and a disease control rate (DCR, defined as CR+PR+SD) was observed in 28 patients (96.6%; “efficacy population, Part D”) (Table 14). A partial response (PR) was confirmed in 10 patients (34.5%), and stable disease (SD) was observed in 18 patients (62.1%). The clinical benefit, defined as patients having CR, PR or prolonged SD (stable disease for 24 weeks or more), was observed in 22 patients (CBR=75.9%).

### Conclusion

The results from the expansion cohort show that combination treatment with 200 mg amcenestrant and palbociclib exhibited a favorable overall safety profile, and demonstrated encouraging antitumor activity in women with endocrine-resistant, ER+/HER2− advanced/metastatic breast cancer (ORR of 34.5% and CBR of 75.9%).

### Example 4: Pooled Population of Parts C and D

The safety analysis was conducted on all patients (“pooled population safety”; n=39) who received at least one dose of the combination of amcenestrant 200 mg with palbociclib (9 and 30 patients in Part C and D, respectively).

The anti-tumor activity of combination amcenestrant/palbociclib treatment was investigated in all patients of Part D and in patients of Part C naïve from the prior targeted therapies with CDK4/6 and mTOR inhibitors, designated hereafter as “Part C naïve patients”. Patients should have progressed during adjuvant therapy or after therapy for metastatic breast cancer. The antitumor activity analysis was based on RECIST1.1 criteria in 35 evaluable patients who were treated with 200 mg amcenestrant with palbociclib combination (“pooled population activity”: 6 patients from Part C (Part C naïve patients) and 29 patients from Part D).

The demographic of the patients treated in the “pooled population safety,” of the study are provided in Table 12.

TABLE 12
Demographic Characteristics of Patient Population

| | Safety Population | |
|---|---|---|
| | Amcenestrant 200 mg + Palbociclib | |
| | (Parts C + D; N = 39) | |
| Age, years, median (range) | 59 | (33-86) |
| ECOG performance score | 29 | (74.4) |
| of 0, n (%) | | |
| Metastatic, n (%) | 35 | (89.7) |

Endocrine resistance status, n (%)

| Primary resistance^{a} | 5 | (12.8) |
|---|---|---|
| Secondary resistance^{b} | 33 | (84.6) |
| Sensitive^{c} | 0 | |
| Unknown | 1 | (2.6) |

Immediate prior therapy, n (%)

| | | |
|---|---|---|
| Neoadjuvant or adjuvant | 16 | (41.0) |
| Advanced | 23 | (59.0) |

Prior lines of therapy in advanced settings, n (%)

| | | |
|---|---|---|
| 0 | 16 | (41.0) |
| 1 line | 15 | (38.5) |
| 2 lines | 6 | (15.4) |
| ≥3 lines | 2 | (5.1) |

Prior lines of endocrine therapy in advanced settings, n (%)

| | | |
|---|---|---|
| 0 | 17 | (43.6) |
| 1 line | 16 | (41.0) |
| 2 lines | 5 | (12.8) |
| ≥3 lines | 1 | (2.6) |

Prior types of anti-cancer treatment in advanced settings, n (%)

| | | |
|---|---|---|
| Prior chemotherapy | 9 | (23.1) |
| Prior endocrine therapy | 23 | (59.0)^{d} |
| Aromatase inhibitors | 18 | (46.2) |
| SERM | 5 | (12.8) |
| SERD (fulvestrant) | 3 | (7.7) |
| Prior targeted therapy | 6 | (15.4) |
| CDK4/6i | 2 | (5.1) |
| Anti-HER2 agents | 1 | (2.6) |
| mTORi | 1 | (2.6) |
| Other | 2 | (5.1) |

^{a}For patients without prior advanced ET, relapse <24 months after the start of adjuvant ET; for patients with prior advanced ET, progression <6 months after the start of the last prior advanced ET.
^{b}For patients without prior advanced ET, relapse ≥24 months after the start and <12 months after the end of adjuvant ET; for patients with prior advanced ET, progression ≥6 months after start of the last prior advanced ET.
^{c}Relapse ≥12 months after the end of adjuvant ET and treatment-naïve in advanced therapy.
^{d}One patient who started on first-line (1L) advanced ET did not have any documented progression and was considered as having zero lines of prior advanced ET.

Characteristic of “Pooled Population Safety” Patients

Among the patients evaluated for the “pooled population safety” (N=39), the mean age was 59.0 (33-86) years. The ECOG status was 0 in 74.4% of the patients, and the remainder of the patients had an ECOG status of 1. The median time from first diagnosis to first study treatment administration was 6.52 years (range of 1.7 to 29.5 years). All patients were ER positive and HER2 negative; 83.8% (31/37) of the patients were PR positive. Patients had a median of two organs involved at study entry (range of one to six); the main organs involved were bone (48.7%), lymph nodes (46.2%), lung (41.0%), liver (35.9%) and pleura (17.9%).

A total of 23/39 (59.0%) patients had received at least one prior anti-cancer treatment in an advanced setting, including 9/39 (23.1%) who received a prior chemotherapy, 6/39 (15.4%) prior targeted therapy (CDK4/6 inhibitor treatment in 2 patients and prior mTOR inhibitor treatment in 1 patient). A total of 23/39 (59.0%) patients had received prior hormonotherapy: 18/39 (46.2%) had received an AI treatment, 3 (7.7%) a SERD-based therapy, and 5/39 (12.8%) a SERM treatment. The remaining 16 patients (41.0%) recurred (relapsed) during the adjuvant therapy while receiving AI or SERM.

Exposure—“Pooled Population Safety”

A total of 29/39 (66.7%) patients underwent at least eight cycles of treatment. The median duration of treatment for the combination therapy was 36 weeks (1-72). A total of 13/39 (33.3%) patients discontinued treatment beforehand with 38/39 (97.4%), 33/39 (84.6%), and 29/39 (74.4%) remaining on treatment at Cycles 2, 4, and 6, respectively. As of Mar. 30, 2021, 23/39 patients (59%) remain on study treatment (Part C: n=2; Part D: n=21).

The median relative dose intensity (RDI) was 99.6% for amcenestrant and about 97.5% for palbociclib. No dose reduction of amcenestrant was reported; ten patients (25.6%) had one dose reduction of palbociclib from 125 to 100 mg, and four patients (10.3%) had one dose reduction of palbociclib from 100 to 75 mg. Four (10.3%) and eleven (28.2%) patients had ≥7 consecutive days of dose omission of amcenestrant and palbociclib, respectively. Twenty-eight (71.8%) patients had at least one temporary dose omission: 48.7% for amcenestrant and 71.8% for palbociclib. A total of sixteen patients (41.0%) permanently discontinued the study treatment, including twelve (30.8%) due to progressive disease.

Safety—“Pooled Population Safety”

The overall safety profile of the “Pooled Population Safety” is presented in Table 13 (TEAE: Treatment emergent adverse event; SAE: Serious Adverse Event). A total of 36 patients (92.3%) experienced at least one TEAE. The most frequently reported non-hematological TEAEs by MedDRA PT, regardless of the relationship to amcenestrant or palbociclib were: fatigue (33.3%), nausea (30.8%), upper abdominal pain, arthralgia, vertigo (17.9%, each), constipation, diarrhea, vomiting, dry skin, asthenia (15.4%, each), dizziness, headache, cough, stomatitis (12.8%, each), urinary tract infection, dysgeusia, hot flush, abdominal pain, pain in extremity (10.3% each). Most of these TEAEs were of grade 1 and 2. TEAEs with Grade ≥3 were reported in a total of 21/39 (53.8%) patients. The most frequently reported non-hematological TEAEs Grade ≥3, regardless of the relationship to amcenestrant or palbociclib, were alanine aminotransferase increase (5.1%). Other TEAEs accounted for less than 5% of all observed TEAEs Grade ≥3.

TABLE 13
Treatment Emergent Adverse Events

| | Amcenestrant 200 mg + Palbociclib 125 mg | | | | | |
|---|---|---|---|---|---|---|
| | PART C | | PART D | | All | |
| n (%)* | (N = 9) | | (N = 30) | | (N = 39) | |
| Participants with any TEAE | 9 | (100) | 27 | (90.0) | 36 | (92.3) |
| Participants with any grade ≥3 TEAE | 6 | (66.7) | 15 | (50.0) | 21 | (53.8) |
| Participants with any grade 3-4 TEAE | 6 | (66.7) | 15 | (50.0) | 21 | (53.8) |
| Participants with any grade 5 TEAE | 0 | | 0 | | 0 | |
| occurring during the treatment period | | | | | | |
| Participants with any treatment emergent | 2 | (22.2) | 5 | (16.7) | 7 | (17.9) |
| SAE | | | | | | |
| Participants with any TEAE related to | 9 | (100) | 19 | (63.3) | 28 | (71.8) |
| Amcenestrant | | | | | | |
| Participants with any TEAE related to | 9 | (100) | 26 | (86.7) | 35 | (89.7) |
| Palbociclib | | | | | | |
| Participants with any TEAE leading to | 1 | (11.1) | 2 | (6.7) | 3 | (7.7) |
| definitive treatment discontinuation | | | | | | |

| | | | |
|---|---|---|---|
| Participants with any TEAE leading to | 0 | 0 | 0 |
| premature discontinuation of palbociclib | | | |

*n(%) = number and percentage of participants with at least one TEAE

28 patients (71.8%) had TEAEs related to amcenestrant and 35 patients (89.7%) had TEAE related to palbociclib. The most frequently reported amcenestrant-related non-hematological TEAEs by MedDRA PT, occurring in at least three patients, were of Grade 1 or 2: nausea and fatigue (170.9%, each), arthralgia, asthenia and hot flushes (10.3%, each), diarrhea, dysgeusia, dry skin and gastroesophageal reflux disease (7.7%). The most frequently reported palbociclib-related non-hematological TEAEs related to by MedDRA PT, occurring in at least three patients, were fatigue (30.8%), nausea (25.6%), asthenia, dysgeusia and stomatitis (10.3%, each), diarrhea, dizziness, dry skin, abdominal pain upper gastroesophageal reflux disease and dry mouth (7.7%, each).

A total of 6 (15.4%) patients had at least one Grade ≥3 TEAE related to amcenestrant, including non-hematological one: febrile neutropenia in 1 (2.6%) patient, and deep vein thrombosis in one (2.6%) patient. A total of 18 (46.2%) patients had at least one Grade ≥3 TEAE related to palbociclib, including non-hematological one: ALT increase in two (5.1%) patients, febrile neutropenia in one (2.6%) patient.

In the pooled safety population, ten serious TEAEs were reported in seven patients (17.9%). Only one of them, deep vein thrombosis, was determined to be related to amcenestrant treatment. A total of five patients (12.8%) presented SAEs of grade ≥3: pneumonia and pleurisy in one patient, duodenal obstruction in one patient, large intestinal obstruction in one patient, gastritis and small intestinal obstruction in one patient, and deep venous thrombosis and back pain in one patient. Additionally, in Part C, one patient who received amcenestrant 200 mg QD in combination with palbociclib 125 mg QD had Grade 2 COVID-19 which occurred during the coronavirus pandemic.

Further details of the non-hematological amcenestrant TRAEs and non-hematological palbociclib TRAEs of “pooled population safety” patients are provided in Table 7 and Table 8, respectively.

The main hematological laboratory abnormalities are as follows. There was no Grade ≥3 anemia. Neutrophil count decreased occurred in 37 patients (94.9%): Grade 1 (5.1%), Grade 2 (33.3%), Grade 3 (48.7%) and Grade 4 (7.7%). For reference, in a pooled analysis conducted in 1666 patients treated with palbociclib, 95.2% of patients had neutrophil count decrease, and grade 1, 2, 3 and 4 neutrophil count decreases were observed in 4.4%, 23.8%, 56.4% and 10.5%, respectively (Palbociclib IB June 2019, Table 6.2-13). Platelet count decreased was observed in 28 patients 71.8% all of Grade 1 except 2 patients with Grade 2. White blood cell decreased was observed in 38 patients (97.4%): Grade 1 (10.3%), Grade 2 (53.8%), Grade 3 (33.3%). Lymphocyte count decreased occurred in 26 patients (66.7%): Grade 1 (17.9%), Grade 2 (30.8%), Grade 3 (15.4%) and Grade 4 (2.6%).

Liver function tests abnormalities were observed as: i) AST increased in 23 patients (59.0%), all Grade 1 except one Grade 2 and one Grade 3; (ii) ALT increased in 19 patients (48.7%), all Grade 1 except one Grade 2 and two Grade 3 and; (iii) blood bilirubin increase: one Grade 3 in one patient (2.6%).

In the pooled safety population, three patients (7.7%) treated with amcenestrant 200 mg QD and palbociclib 125 mg QD 3w/1w dropped out of the study treatment: one patient with Grade 2 asthenia during treatment cycle 3 related to both compounds, one patient with Grade 3 large intestinal obstruction during cycle 5 not related to treatments, and one patient with Grade 3 ALT increased during cycle 2 related to palbociclib.

No patient died during the treatment period, and no patient died after the treatment period due to disease progression.

Antitumor Activity—“Pooled Population Activity”

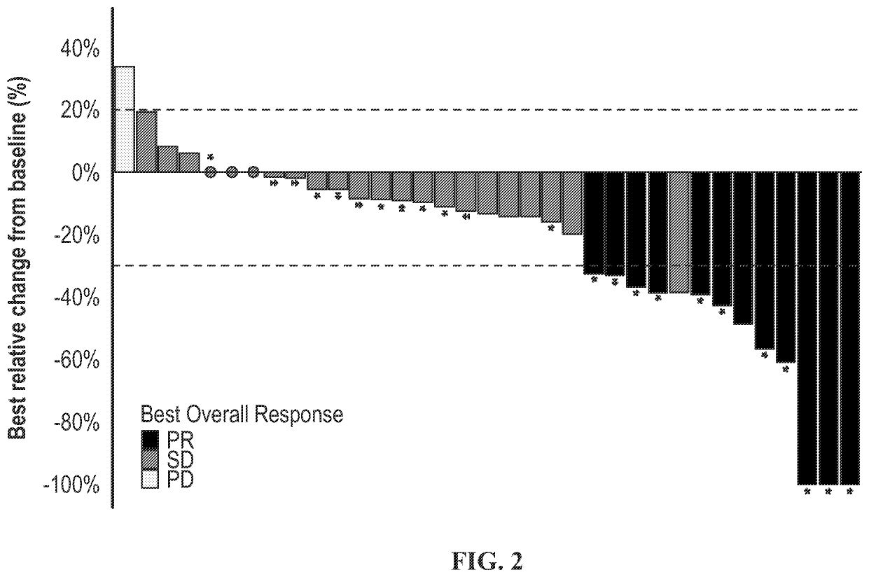

The antitumor activity of the combination of palbociclib with amcenestrant 200 mg was assessed in 35 evaluable patients naïve from the targeted therapies as described above (“pooled population activity”: 6/9 patients from Part C and 29 from Part D) based on RECIST1.1 criteria (Table 14, FIG. 2). The best overall response (BOR) was as follows: 12 confirmed PR (34.3%), 22 SD (62.9%), and one PD (2.9%). The objective response was seen in 12/35 patients (34.3%). The disease control rate (DCR: CR+PR+SD) was 97.2% and the clinical benefit was observed in 26/35 patients (74.3%). Tumor shrinkage has been observed in 80% of the patients, including three patients showing 100% shrinkage of target lesions. No change from baseline in tumor size has been seen in 3/35 (8.6%) of the patients (FIG. 2). The clinical benefit was observed in 7/8 patients with ESR1 mutations (CBR=87.5%) and in 19/27 patients with wild-type ESR1 (CBR=70.4%).

TABLE 14
Best overall response

| | Amcenestrant 200 mg + Palbociclib 125 mg | | |
|---|---|---|---|
| | PART C | PART D | All |
| | (N = 6) | (N = 29) | (N = 35) |
| Best Overall Response [n(%)] | | | |
| Number | 6 | 29 | 35 |
| Complete Response (CR)^{a} | 0 | 0 | 0 |
| Partial Response (PR)^{a} | 2 (33.3) | 10 (34.5) | 12 (34.3) |
| Stable Disease (SD) | 4 (66.7) | 18 (62.1) | 22 (62.9) |
| Progressive Disease (PD) | 0 | 1 (3.4) | 1 (2.9) |
| Objective Response Rate | 2 (33.3) | 10 (34.5) | 12 (34.3) |
| (confirmed CR and PR) [n(%)] | | | |
| 90% CI^{b} | (6.3%, 72.9%) | (20.0%, 51.4%) | (21.1%, 49.6%) |
| Clinical Benefit Rate [n(%)]^{c} | 4 (66.7) | 22 (75.9) | 26 (74.3) |
| 90% CI^{b} | (27.1%, 93.7%) | (59.4%, 88.1%) | (59.4%, 85.9%) |

^{a}Confirmation of response is required (i.e. a second examination done at least 4 weeks apart [i.e. >=28 days], in order to confirm the antitumoral response)
^{b}Estimated by Clopper Pearson interval
^{c}Clinical Benefit Rate: CR, PR or prolonged SD (meaning stable disease for 24 weeks or more)

The ORR, DCR and CBR of combination treatment with amcenestrant and palbociclib (AMEERA-1 study) were 34%, 97% and 74%. For reference, the ORR, DCR and CBR of combination treatment with fulvestrant and palbociclib (PALOMA-3 study) were 25%, 78% and 64%, respectively (Table 15).

TABLE 15
Antitumor activity in AMEERA-1 and PALOMA-3 studies

| Trial | Study regimen | N | ORR | CBR | DCR |
|---|---|---|---|---|---|
| AMEERA-1 | Amcenestrant + palbociclib | 35 | 34% | 74% | 97% |
| PALOMA-3⁽¹⁾ | Fulvestrant + palbociclib | 268 | 25% | 64% | 78% |

⁽¹⁾Cristofanilli et al., Lancet Oncology. (2016) 17: 425-39.

### Conclusion

The analysis of the combination treatment with 200 mg amcenestrant with palbociclib in the “pooled population safety” patients indicates a favorable safety profile. The TEAEs related to amcenestrant observed in the combination therapy were similar to those observed with amcenestrant monotherapy. The incidence of neutropenia or neutrophil count decrease related to palbociclib was in line with the pooled safety data of palbociclib combined with endocrine therapy described in the literature.

The antitumor activity of the combination therapy at the RD of amcenestrant (200 mg QD) with the standard approved dose of palbociclib (125 mg QD, 3w/1w) observed in hormonal resistant patients with ER+/HER2− advanced/metastatic breast cancer, without prior treatment with a CDK4/6 or mTOR inhibitor, is very encouraging, with a clinical benefit rate observed of 74.3%.

### Example 5: Updated Clinical Trial Results for the Response-Evaluable Population

This Example describes clinical trial results from patient database extraction with a cut-off date of 30 May 2021. Table 16 shows baseline demographics and disease characteristics in the response-evaluable population, with a total number of 34 patients (Parts C and D of the trial).

TABLE 16
Baseline patient demographics and disease characteristics

| Demographics and Disease | Response-Evaluable Population | |
|---|---|---|
| Characteristics | (Parts C + D; N = 34) | |
| Age, years, median (range) | 61 | (33-86) |
| ECOG performance score | 27 | (79.4) |
| of 0, n (%) | | |
| Metastatic, n (%) | 31 | (91.2) |

Endocrine resistance status, n (%)

| | | |
|---|---|---|
| Primary resistance^{a} | 3 | (8.8) |
| Secondary resistance^{b} | 31 | (91.2) |

| | |
|---|---|
| Sensitive^{c} | 0 |

Immediate prior therapy, n (%)

| | | |
|---|---|---|
| Neoadjuvant or adjuvant | 15 | (44.1) |
| Advanced | 19 | (55.9) |

Prior lines of therapy in advanced settings, n (%)

| | | |
|---|---|---|
| 0 | 15 | (44.1) |
| 1 line | 14 | (41.2) |
| 2 lines | 5 | (14.7) |

| | |
|---|---|
| ≥3 lines | 0 |

Prior lines of endocrine therapy in the advanced setting, n (%)

| | | |
|---|---|---|
| 0 | 16 | (47.1) |
| 1 line | 15 | (44.1) |
| 2 lines | 3 | (8.8) |

| | |
|---|---|
| ≥3 lines | 0 |

Prior types of anti-cancer treatment in the advanced setting, n (%)

| | | |
|---|---|---|
| Prior chemotherapy | 6 | (17.6) |
| Prior endocrine therapy | 18 | (52.9) |
| Aromatase inhibitors | 14 | (41.2) |
| SERM | 5 | (14.7) |
| SERD (fulvestrant) | 1 | (2.9) |
| Prior targeted therapy | 2 | (5.9) |

^{a}Relapse while on the first two years of adjuvant ET, or PD within the first six months of first-line ET for aBC while on ET.
^{b}Relapse while on adjuvant ET but after the first two years, or relapse within twelve months of completing adjuvant ET, or PD ≥six months after initiating ET for aBC while on ET.
^{c}Relapse ≥twelve months after the end of adjuvant ET and treatment-naïve in advanced therapy.

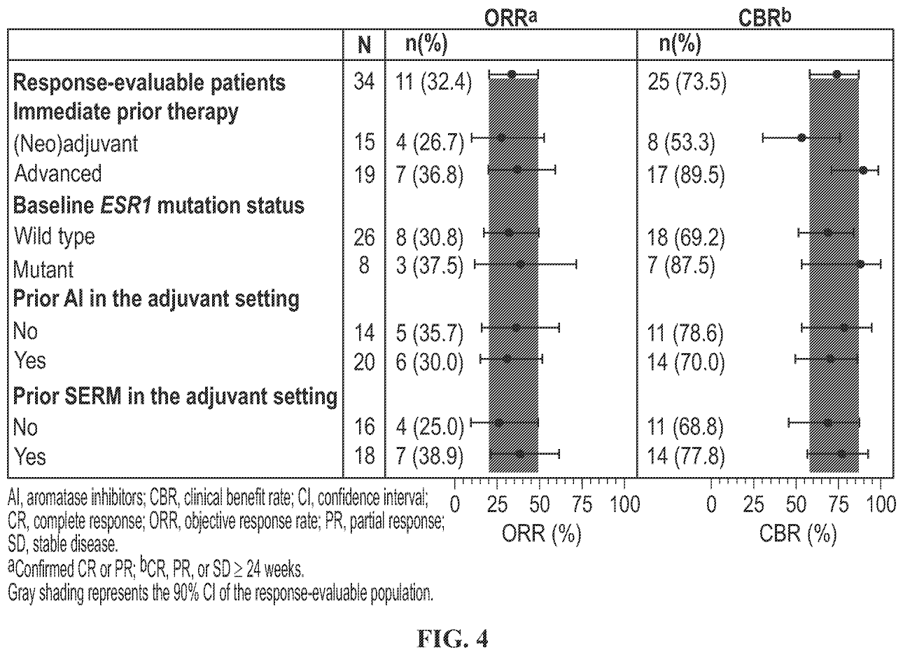

FIG. 4 shows antitumor activity in this response-evaluable population (34 patients) overall and by subgroups depending on prior therapy and baseline ESR1 mutation status.

Tables 17 and 18 show the objective response and clinical benefit, respectively, in the response-evaluable patients (N=34) with available data for baseline ESR1 mutational analyses in cfDNA by ddPCR.

TABLE 17
Objective response in the response-evaluable patients

| | Nonresponders | Responders | |
|---|---|---|---|
| ESR1 Mutations | (N = 23) | (N = 11) | |
| Patients with wildtype ESR1, n | 18 | 8 | |
| Patients with mutated ESR1, n | 5 | 3 | |
| D538G, n (%) | 1 (20.0) | 1 | (33.3) |
| E380Q, n (%) | 1 (20.0) | 2 | (66.7) |
| Y537N, n (%) | 1 (20.0) | 0 | (0) |
| Y537S, n (%) | 2 (40.0) | 0 | (0) |

TABLE 18
Clinical benefit in the response-evaluable patients

| | No clinical benefit | Clinical benefit |
|---|---|---|
| ESR1 Mutations | (N = 9) | (N = 25) |
| Patients with wildtype ESR1, n | 8 | 18 |
| Patients with mutated ESR1, n | 1 | 7 |
| D538G, n (%) | 0 (0) | 2 (28.6) |
| E380Q, n (%) | 1 (100) | 2 (28.6) |
| Y537N, n (%) | 0 (0) | 1 (14.3) |
| Y537S, n (%) | 0 (0) | 2 (28.6) |

In response-evaluable patients with available data for baseline ESR1 mutational analyses in cfDNA by ddPCR (N=34), objective response (OR) was observed in 3/8 (37.5%) patients with ESR1 mutations (two patients had an E380Q mutation and one patient had a D538G mutation) and in 8/26 (30.8%) patients with wildtype ESR1 (Table 17). Clinical benefit (CB) was observed in 7/8 (87.5%) patients with baseline ESR1 mutations (two with D538G, two with E380Q, one with Y537N, and two with Y537S), and in 18/26 (69.2%) patients with wildtype ESR1 (Table 18).

### Conclusion

The data presented in this Example confirm the conclusions drawn in Examples 1-4 above. Among patients with endocrine-resistant ER+/HER2− advanced/metastatic breast cancer, amcenestrant 200 mg QD combined with the standard approved dose of palbociclib demonstrated encouraging antitumor activity, including in patients with tumors with baseline ESR1 mutations.

### Example 6: Updated Clinical Trial Results, Including PFS Results and Genomic Data

This Example describes an update of the safety and antitumor activity results, progression-free survival (PFS) results, and genomic data from patient database extraction with a cut-off date of 4 Oct. 2021. Table 19 shows baseline demographic and disease characteristics in the safety population, with a total number of 39 patients. All patients had prior endocrine resistance.

TABLE 19
Baseline demographic and disease characteristics

| | Safety Population | |
|---|---|---|
| | Amcenestrant 200 mg + | |
| | Palbociclib 125 mg | |
| | (Parts C + D; N = 39) | |
| Age, years, median (range) | 59 | (33-86) |
| ECOG performance score | 29 | (74.4) |
| of 0, n (%) | | |
| Metastatic, n (%) | 35 | (89.7) |

Endocrine resistance status, n (%)

| | | |
|---|---|---|
| Primary resistance^{a} | 6 | (15.4) |
| Secondary resistance^{b} | 33 | (84.6) |

| | |
|---|---|
| Sensitive^{c} | 0 |

Intent of last prior therapy, n (%)

| | | |
|---|---|---|
| Neoadjuvant or adjuvant | 14 | (35.9) |
| Advanced | 25 | (64.1) |

Prior lines of therapy in advanced settings, n (%)

| | | |
|---|---|---|
| 0 | 14 | (35.9) |
| 1 line | 16 | (41.0) |
| 2 lines | 7 | (17.9) |
| 3 lines | 1 | (2.6) |
| >3 lines | 1 | (2.6) |

Prior lines of endocrine therapy in advanced settings, n (%)

| | | |
|---|---|---|
| 0 | 15 | (38.5) |
| 1 line | 17 | (43.6) |
| 2 lines | 6 | (15.4) |
| ≥3 lines | 1 | (2.6) |

Prior types of anti-cancer treatment in advanced settings n (%)

| | | |
|---|---|---|
| Prior chemotherapy | 10 | (25.6) |
| Prior endocrine therapy | 24 | (61.5) |
| Aromatase inhibitors | 19 | (79.2) |
| SERM | 6 | (25.0) |
| SERD | 3 | (12.5) |
| Other | 1 | (4.2) |
| Prior targeted therapy | 6 | (15.4) |
| CDK4/6i | 2 | (33.3) |
| Anti-HER2 agents | 1 | (16.7) |
| mTORi | 2 | (33.3) |
| Other | 2 | (33.3) |

^{a}For patients without prior advanced ET, relapse <24 months after the start of adjuvant ET; for patients with prior advanced ET, progression <6 months after the start of the last prior advanced ET.
^{b}For patients without prior advanced ET, relapse ≥24 months after the start and <12 months after the end of adjuvant ET; for patients with prior advanced ET, progression ≥6 months after start of the last prior advanced ET.
^{c}Relapse ≥12 months after the end of adjuvant ET and treatment-naïve in advanced therapy.

As of Oct. 4, 2021, 16/39 (41.0%) patients remained on study treatment (all in Part D). More than half of all patients in the safety population (22/39, 56.4%) initiated at least 14 cycles (56 weeks) of treatment. The median (range) duration of treatment was 57.0 (1-98) weeks.

The median relative dose intensity (RDI) was 99.6% for amcenestrant and 97.4% for palbociclib. Amcenestrant dose reduction occurred in one (2.6%) patient. Thirteen (33.3%) patients had ≥one palbociclib dose reduction with reduction to 100 mg, and four (10.3%) of these patients had a subsequent reduction to 75 mg. Eight (20.5%) and 17 (43.6%) patients had ≥7 consecutive days of dose omission of amcenestrant and palbociclib, respectively.

Safety was evaluated in the “safety population,” defined as patients receiving amcenestrant 200 mg in combination with palbociclib (N=39: Part C, n=9; Part D, n=30). Safety assessments were based upon treatment-related adverse events (TRAEs) coded according to the Medical Dictionary for Regulatory Activities (MedDRA) v23.1. Neutrophil count decrease was reported based on hematological laboratory abnormalities per the Common Terminology Criteria for Adverse Events (CTCAE) v4.03 during the on-treatment period. Table 20 shows the non-hematological TRAEs occurring in ≥10% of patients in the safety population.

TABLE 20
Non-hematological TRAEs occurring in
≥10% of patients (safety population)

| | Safety Population | |
|---|---|---|
| | Amcenestrant 200 mg + Palbociclib | |
| | (Parts C + D, N = 39) | |
| | All Grades | Grade ≥3 |
| Amcenestrant Non-hematological | | |
| TRAEs, n (%) | | |
| Nausea | 7 (17.9) | 0 |
| Fatigue | 6 (15.4) | 0 |
| Arthralgia | 4 (10.3) | 0 |
| Asthenia | 4 (10.3) | 0 |
| Dry skin | 4 (10.3) | 0 |
| Hot flush | 4 (10.3) | 0 |
| Palbociclib Non-hematological | | |
| TRAEs, n (%) | | |
| Fatigue | 12 (30.8) | 0 |
| Nausea | 10 (25.6) | 0 |
| Asthenia | 4 (10.3) | 0 |
| Dry skin | 4 (10.3) | 0 |
| Dysgeusia | 4 (10.3) | 0 |
| Stomatitis | 4 (10.3) | 0 |

Three (7.7%) patients discontinued treatment due to adverse events: two were related to treatment (one amcenestrant and palbociclib [asthenia]; one palbociclib only [alanine aminotransferase elevation]), and one was not related to treatment (large intestinal obstruction).

TRAEs related to amcenestrant and palbociclib, respectively, occurred in 27/39 (69.2%) and 36/39 (92.3%) patients for all grade events and in 5/39 (12.8%) and 19/39 (48.7%) patients for Grade ≥3 events. Neutrophil count decrease based on hematological laboratory abnormalities was observed in the majority of patients (all grades, 94.9%; Grade 3, 43.6%; Grade 4, 12.8%). The most common treatment-related adverse events for amcenestrant and palbociclib were Grade 1 or 2 nausea and fatigue. Grade 3 amcenestrant-related serious adverse events of deep vein thrombosis and thrombosis occurred in one patient (2.6%). No clinically significant cardiac or ocular safety findings or deaths occurred in the study period.

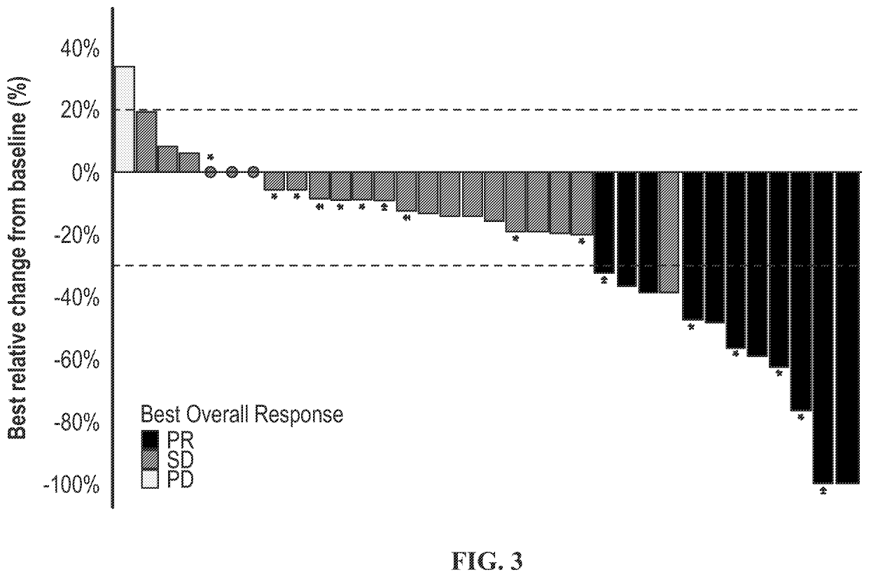

Antitumor activity was evaluated in the “response-evaluable population,” composed of patients without prior CDK4/6i or mTORi treatment, who received amcenestrant 200 mg in combination with palbociclib (N=34: Part C, n=5; Part D, n=29). Antitumor activity was assessed according to the Response Evaluation Criteria in Solid Tumors (RECIST) v1.1 per investigators/local radiologists using the objective response rate (ORR; confirmed complete response [CR] and confirmed partial response [PR]), clinical benefit rate (CBR; CR, PR, or stable disease [SD]≥24 weeks), and PFS (time interval from the first treatment intake to the date of the first tumor progression or death). Table 21, Table 22, and FIG. 3 show the antitumor activity results in the response-evaluable population.

TABLE 21
Antitumor activity results in the response-evaluable population

| | Amcenestrant 200 mg + Palbociclib 125 mg | | |
|---|---|---|---|
| | PART C | PART D | All |
| | (N = 5) | (N = 29) | (N = 34) |
| Best Overall Response [n(%)] | | | |
| Number | 5 | 29 | 34 |
| Complete Response (CR)^{a} | 0 | 0 | 0 |
| Partial Response (PR)^{a} | 1 (20.0) | 10 (34.5) | 11 (32.4) |
| Stable Disease (SD) | 4 (80.0) | 18 (62.1) | 22 (64.7) |
| Progressive Disease (PD) | 0 | 1 (3.4) | 1 (2.9) |
| Objective Response Rate | 1 (20.0) | 10 (34.5) | 11 (32.4) |
| (confirmed CR and PR) [n(%)] | | | |
| 90% CI^{b} | (1.0%, 65.7%) | (20.0%, 51.4%) | (19.3%, 47.8%) |
| Clinical Benefit Rate [n(%)]^{c} | 3 (60.0) | 22 (75.9) | 25 (73.5) |
| 90% CI^{b} | (18.9%, 92.4%) | (59.4%, 88.1%) | (58.4%, 85.4%) |

^{a}Confirmation of response is required (i.e. a second examination done at least 4 weeks apart [i.e. >=28 days], in order to confirm the antitumoral response).
^{b}Estimated by Clopper Pearson interval.
^{c}Clinical Benefit Rate: CR, PR or prolonged SD (meaning stable disease for 24 weeks or more).

TABLE 22
PFS results in the response-evaluable population

| | Response-evaluable Population | |
|---|---|---|
| | (Parts C + D; N = 34) | |
| Median follow-up^{a}, months | 14.8 | |
| Median PFS [90% CI]^{b}, months | 14.7 | [11.0, 22.3] |
| Number of events, n (%) | 17 | (50.0%) |
| Number of censoring, n (%) | 17 | (50.0%) |
| PFS rate (%) at 12 months [90% CI] | 59.4 | [43.8%, 72.0%] |

^{a}Based on the reverse Kaplan-Meier method;
^{b}Kaplan-Meier estimates. CIs are computed using the log-log method.

In terms of activity in the response-evaluable population, the objective response rate was 32.4%, and the clinical benefit rate was 73.5%. Median time to first response was 16.3 weeks (range: 8-32 weeks). Tumor shrinkage was observed in 27/34 (79.4%) patients, with two patients showing 100% shrinkage of their target lesions. After a median follow-up of 14.8 months, the median progression-free survival was 14.7 months.

For reference, in the phase 3 PALOMA-3 study evaluating combination treatment with fulvestrant and palbociclib in patients with HR+/HER2− metastatic breast cancer (521 patients with progressive disease after endocrine therapy and ≤1 prior chemotherapy for advanced breast cancer) versus fulvestrant and placebo, a median PFS of 9.5 months was achieved with the combination treatment (Cristofanilli et al., supra).

Genomic data were analyzed in plasma circulating free DNA (cfDNA) by droplet digital polymerase chain reaction (ddPCR) in twelve pathogenic, independent ESR1 mutations using the OncoBEAM™ platform by Sysmex Inostics (Baltimore, MD, USA) and by next-generation sequencing (NGS) using the Roche AVENIO extended panel (77-gene) by Ambry Genetics (Aliso Viejo, CA, USA):
ESR1 mutational profiling by ddPCR in patients with available data:
   at baseline (Cycle 1 Day 1, C1D1) in the all-treated population (N=45),
   at baseline and on treatment (Cycle 2 Day 2, C2D28) in the all-treated population (N=31);
   mutational profiling by NGS on 77 genes, including ESR1, in patients with available data at baseline in the all-treated population (N=45).

The all-treated population is the number of exposed patients. Among patients in the all-treated population whose most recent prior anticancer therapy was in the advanced setting, 13/30 (43.3%) patients had at least one ESR1 mutation at baseline. The most frequent mutations besides ESR1 were PI3KCA and TP53 (33% each). The observations are consistent with those made for patients treated with amcenestrant monotherapy.

Of 15 patients in the all-treated population whose most recent prior anticancer therapy was in the (neo)adjuvant setting, only 1/15 (6.7%) had an ESR1 mutation. Among patients in the all-treated population with available ESR1 mutation data by ddPCR at baseline and on treatment (N=31), 5/9 (55.6%) patients with baseline mutated ESR1 had wild-type ESR1 at C2D28, with clearance of mutations including D538G, E380Q, and Y537S; one patient had a new mutation (Y537C) detected at C2D28. Among most patients with an ESR1 mutation at baseline, mutated ESR1 allele frequency decreased from baseline to Cycle 2, Day 28.

### Conclusion

As shown herein, among postmenopausal women with endocrine-resistant ER+/HER2− advanced breast cancer, treatment with amcenestrant 200 mg in combination with the standard approved dose of palbociclib resulted in a median progression-free survival of 14.7 months at a median follow-up of 14.8 months, and demonstrated antitumor activity (ORR: 32.4%; CBR: 73.5%) and a favorable safety profile, consistent with previous results. In addition, this combination treatment exhibited a reduction of several ESR1 mutations in cfDNA after two cycles of treatment, including the traditionally resilient D538G and Y537S mutations.

Overall, it can be concluded from the data presented herein that the 200 mg dose of amcenestrant combined with the CDK4/6i palbociclib (125 mg) demonstrates encouraging safety and antitumor activity, including in terms of progression-free survival, in hormonal resistant patients with ER+/HER2− advanced/metastatic breast cancer that are naïve to prior treatment with a CDK4/6 or mTOR inhibitor.

1. A method of treating breast cancer in a patient in need thereof, comprising orally administering to the patient amcenestrant, or a pharmaceutically acceptable salt thereof, at a dose of 200-400 mg once daily and orally administering to the patient palbociclib, or a pharmaceutically acceptable salt thereof, at a dose of 75-125 mg once daily.

2. The method of claim 1, wherein the daily dose of amcenestrant is 200 mg.

3. The method of claim 1, wherein the daily dose of palbociclib is 75, 100, or 125 mg.

4. (canceled)

5. The method of claim 1, wherein amcenestrant and palbociclib or a pharmaceutically acceptable salt thereof are administered to the patient in one or more treatment cycles.

6. The method of claim 5, wherein the patient is treated with 1, 2, 3, 4, 5, 6, 7, 8, 9, 10, 11, 12, 13, 14 or more treatment cycles.

7. The method of claim 5, wherein each of the one or more treatment cycles comprises or consists of 28 days.

8. The method of claim 7, wherein the patient is administered amcenestrant daily, and is administered palbociclib, or a pharmaceutically acceptable salt thereof, daily for 21 consecutive days only, during each of the one or more treatment cycles.

9. The method of claim 5, wherein a treatment cycle is followed immediately by a subsequent treatment cycle if more than one treatment cycle is administered.

10. The method of claim 1, wherein the patient is treated with amcenestrant daily at 200 mg per day, and with palbociclib at 125 mg daily for 21 consecutive days only, in one or more 28-day treatment cycles.

11. The method of claim 1, wherein the breast cancer is hormone receptor (HR) positive.

12. The method of claim 11, wherein the HR is estrogen receptor (ER).

13. The method of claim 11, wherein the HR is progesterone receptor (PgR).

14. The method of claim 11, wherein the breast cancer is estrogen receptor positive (ER+) and progesterone receptor positive (PgR+).

15. The method of claim 11, wherein the breast cancer is human epidermal growth factor receptor 2 negative (HER2−).

16. The method of claim 15, wherein the breast cancer is ER+ and HER2−.

17. The method of claim 11, wherein the breast cancer is advanced or metastatic cancer.

18. The method of claim 11, wherein the patient is a postmenopausal woman with ER+/HER2− advanced or metastatic breast cancer.

19. The method of claim 11, wherein an ESR1 gene in the patient is mutated.

20. The method of claim 11, wherein the patient is refractory to endocrine therapy.

21. The method of claim 1, wherein the patient has not previously been treated with a CDK4/6 or mTOR inhibitor.

22. The method of claim 1, wherein the method results in partial response (PR) or stable disease (SD) in the patient.

23. The method of claim 1, wherein the method results in progression-free survival, optionally a median progression-free survival of about 14.7 months, in the patient.

24. The method of claim 1, further comprising monitoring the patient for neutropenia or lung inflammation.

25. The method of claim 1, wherein amcenestrant or a pharmaceutically acceptable salt thereof is provided as a capsule or a tablet or palbociclib or a pharmaceutically acceptable salt thereof is provided as a capsule or a tablet.

26. (canceled)

27. An article of manufacture or kit, comprising an amcenestrant capsule or tablet at 200 mg per capsule or tablet and a palbociclib capsule or tablet at 125 mg per capsule or tablet, optionally wherein the article of manufacture or kit comprises 28 of the amcenestrant capsules or tablets and 21 of the palbociclib capsules or tablets, and optionally instructions for use for treating ER+/HER2− breast cancer.

28-32. (canceled)
